(19) United States
(12) Patent Application Publication
(10) Pub. No.: US 2021/0371895 A1
(43) Pub. Date: Dec. 2, 2021

(54) BACTERIAL RESPONSE

(71) Applicants: BioFire Diagnostics, LLC, Salt Lake City, UT (US); bioMerieux SA, Marcy-L'Etoile (FR)

(72) Inventors: Robert J. Crisp, Clayton, MO (US); Andrew Clinton Hemmert, Salt Lake City, UT (US); Stefanie Marxreiter, Salt Lake City, UT (US); Eric Lo, Salt Lake City, UT (US); Laurent Eugène Paul Drazek, Grenoble (FR); Matthew F. Hockin, Salt Lake City, UT (US); Joshua Earle Jackson, Cottonwood Heights, UT (US)

(21) Appl. No.: 17/282,104

(22) PCT Filed: Oct. 1, 2019
(86) PCT No.: PCT/US2019/054015

§ 371 Date: Apr. 1, 2021

Related U.S. Application Data
(60) Provisional application No. 62/739,949, filed on Oct. 2, 2018.

(51) Int. Cl. C12Q 1/18; C12Q 1/6806; C12Q 1/6851

(52) U.S. Cl. CPC C12Q 1/18; B01L 3/502; C12Q 1/6851; C12Q 1/6806

(57) ABSTRACT

Methods, sample vessels, and instruments are provided for determining antibiotic resistance of a bacterium.

### CROSS REFERENCE TO RELATED APPLICATIONS

This application claims the benefit of and priority to U.S. App. Ser. No. 62/739,949 filed Oct. 2, 2018, the entirety of which is incorporated herein by reference.

### BACKGROUND

In the United States, Canada, and Western Europe infectious disease accounts for approximately 7% of human mortality, while in developing regions infectious disease accounts for over 40% of human mortality. Infectious diseases lead to a variety of clinical manifestations. Among common overt manifestations are fever, pneumonia, meningitis, diarrhea, and diarrhea containing blood. While the physical manifestations suggest some pathogens and eliminate others as the etiological agent, a variety of potential causative agents remain, and clear diagnosis often requires a variety of assays to be performed. Traditional microbiology techniques for diagnosing pathogens can take days or weeks, often delaying a proper course of treatment.

In recent years, the polymerase chain reaction (PCR) has become a method of choice for rapid diagnosis of infectious agents. PCR can be a rapid, sensitive, and specific tool to diagnose infectious disease. A challenge to using PCR as a primary means of diagnosis is the variety of possible causative organisms and the low levels of organism present in some pathological specimens. It is often impractical to run large panels of PCR assays, one for each possible causative organism, most of which are expected to be negative. The problem is exacerbated when pathogen nucleic acid is at low concentration and requires a large volume of sample to gather adequate reaction templates. In some cases, there is inadequate sample to assay for all possible etiological agents. A solution is to run “multiplex PCR” wherein the sample is concurrently assayed for multiple targets in a single reaction. While multiplex PCR has proven to be valuable in some systems, shortcomings exist concerning robustness of high level multiplex reactions and difficulties for clear analysis of multiple products. To solve these problems, the assay may be subsequently divided into multiple secondary PCRs. Nesting secondary reactions within the primary product often increases robustness. However, this further handling can be expensive and may lead to contamination or other problems.

Fully integrated multiplex PCR systems integrating sample preparation, amplification, detection, and analysis are user friendly and are particularly well adapted for the diagnostic market and for syndromic approaches. The FilmArray® (BioFire Diagnostics, LLC, Salt Lake City, Utah) is such a system, a user friendly, highly multiplexed PCR system developed for the diagnostic market. The single sample instrument accepts a disposable “pouch” that integrates sample preparation and nested multiplex PCR. Integrated sample preparation provides ease-of-use, while the highly multiplexed PCR provides both the sensitivity of PCR and the ability to test for up to 30 different organisms simultaneously. This system is well suited to pathogen identification where a number of different pathogens all manifest similar clinical symptoms. Current available diagnostic panels include a respiratory panel for upper respiratory infections, a blood culture panel for blood stream infections, a gastrointestinal panel for GI infections, and a meningitis panel for cerebrospinal fluid infections. Other panels are in development.

While the FilmArray instrument has been used for identification of various pathogens from a single sample, the FilmArray and other quantitative and semi-quantitative systems may be suitable for use in detection of antibiotic susceptibility. Antibiotic susceptibility can be measured on a molecular level by detecting transcriptional differences in susceptible and resistant bacteria in response to antibiotic exposure. While these transcriptional differences can be discovered using RNA sequencing or cDNA microarray analysis, the large multiplex and reverse-transcription capabilities systems such as the FilmArray could facilitate measuring antibiotic susceptibility for multiple bacteria and antibiotics.

Resistance to antibiotics is a major public threat, with mortality rates that are an estimated five-fold higher for resistant organisms. By 2050, it is projected that antibiotic resistance will lead to 10 million deaths every year, with a cost of 100 trillion US dollars. Current microbiology methods for antibiotic resistance involve broth microdilution, including plating followed by inoculating broths against various concentrations of antibiotics. The broths are checked for “cloudiness” of the inoculum, or colorimetric changes, either visually or via microscopy. Alternatively, agar dilution may be used, wherein antibiotic dilution is impregnated into agar, bacteria are inoculated onto the agar dilution series, plates are grown, and then are visually inspected for the presence or absence of growth and at which dilution. Other microbiological methods are known, including automated systems, but all require bacterial growth while challenging the bacteria with varying concentrations of different antibiotics. These methods take many hours to several days to complete. Thus, rapid and accurate identification of antibiotic resistance is needed, so that patients may be properly treated in a timely manner.

In one illustrative example, specific and generic bacteria-antibiotic combinations could be targeted, wherein a sample loading vessel with a cocktail of antibiotics could be provided, resulting broad susceptibility results.

In another illustrative example, generic bacteria-antibiotic gene targets are included, and unique sample loading vessels with single antibiotics may be provided, resulting in narrow susceptibility results.

### BRIEF SUMMARY

In one aspect of the present disclosure, methods are provided for determining antibiotic resistance of a bacterium in a sample.

According to an aspect of the present invention a method for determining antibiotic resistance of a bacterium in a sample comprises: (a) incubating the sample with an antibiotic, (b) isolating RNA from the sample, (c) reverse-transcribing the RNA for a plurality of genes that each show a different pattern of expression between susceptible and resistant strains, (d) amplifying targets from the plurality of genes that each show a different pattern of expression between susceptible and resistant strains to generate a plurality of amplified targets, (e) quantifying each of the plurality of amplified targets from the plurality of genes to provide a plurality of quantified amplified targets and to generate a value indicative of antibiotic susceptibility, and (f) determining antibiotic resistance from the value indicative of antibiotic susceptibility.

A further aspect of the present disclosure is directed to a method for determining antibiotic resistance of a bacterium in a sample comprising: (a) incubating the sample with an antibiotic, (b) isolating RNA from the sample, (c) reverse-transcribing the RNA for a gene that shows a different pattern of expression between susceptible and resistant strains, (d) amplifying a target from the gene that shows the different pattern of expression between susceptible and resistant strains to generate an amplified target, (e) quantifying the amplified target to generate a value indicative of antibiotic susceptibility, and (f) determining antibiotic resistance from the value indicative of antibiotic susceptibility.

Another aspect of the present disclosure is directed to a container for determining antibiotic resistance of a bacterium in a sample comprising: a first-stage reaction zone comprising a first-stage reaction blister comprising a plurality of pairs of primers for reverse-transcription and amplification of a plurality of genes that each show a different pattern of expression between susceptible and resistant strains, and a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show a different pattern of expression between susceptible and resistant strains, the second-stage reaction zone configured for thermal cycling all of the plurality of second-stage reaction chambers.

A further aspect of the present invention is directed to a device for analyzing a sample, comprising: an opening configured to receive a container, the container comprising a first-stage reaction zone comprising a plurality of pairs of primers for reverse-transcription and amplification of a plurality of genes that each show a different pattern of expression between susceptible and resistant strains or a reference gene, and a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show the different pattern of expression between susceptible and resistant strains or the reference gene, the plurality of second-stage reaction chambers further comprising a detectable label that produces a signal indicative of an amount of amplification, a first heater for controlling temperature of the first-stage reaction zone, a second heater for thermal cycling the second-stage reaction zone, a detection device configured to detect the signal in each of the second-stage reaction chambers, and a CPU configured to determine a Cp for each of the plurality of genes that each show the different pattern of expression between susceptible and resistant strains and the reference gene, and configured to output a value for each of the plurality of genes that each show the different pattern of expression between susceptible and resistant strains, wherein the value is a ΔCp or absolute value of a ΔCp for each of the plurality of genes that each show the different pattern of expression between susceptible and resistant strains, and wherein the CPU is configured to determine antibiotic resistance from the values for each of the plurality of genes that each show the different pattern of expression between susceptible and resistant strains.

An additional aspect of the present invention is directed to use of a container as described herein, optionally use of the container in a method as described herein (e.g., a method for determining antibiotic resistance of a bacterium in a sample). In some embodiments, the container comprises: a first-stage reaction zone comprising a first-stage reaction blister comprising a plurality of pairs of primers for reverse-transcription and amplification of a plurality of genes that each show a different pattern of expression between susceptible and resistant strains, and a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show a different pattern of expression between susceptible and resistant strains, the second-stage reaction zone configured for thermal cycling all of the plurality of second-stage reaction chambers.

Another aspect of the present invention is directed to use of a device as described herein, optionally use of the device in a method as described herein (e.g., a method for determining antibiotic resistance of a bacterium in a sample). In some embodiments, the device comprises: an opening configured to receive a container, the container comprising a first-stage reaction zone comprising a plurality of pairs of primers for reverse-transcription and amplification of a plurality of genes that each show a different pattern of expression between susceptible and resistant strains or a reference gene, and a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show the different pattern of expression between susceptible and resistant strains or the reference gene, the plurality of second-stage reaction chambers further comprising a detectable label that produces a signal indicative of an amount of amplification, a first heater for controlling temperature of the first-stage reaction zone, a second heater for thermal cycling the second-stage reaction zone, a detection device configured to detect the signal in each of the second-stage reaction chambers, and a CPU configured to determine a Cp for each of the plurality of genes that each show the different pattern of expression between susceptible and resistant strains and the reference gene, and configured to output a value for each of the plurality of genes that each show the different pattern of expression between susceptible and resistant strains, wherein the value is a ΔCp or absolute value of a ΔCp for each of the plurality of genes that each show the different pattern of expression between susceptible and resistant strains, and wherein the CPU is configured to determine antibiotic resistance from the values for each of the plurality of genes that each show the different pattern of expression between susceptible and resistant strains.

A further aspect of the present invention is directed to a method for determining the minimal inhibitory concentration (MIC) of an antibiotic towards a bacterium in a sample comprising: incubating an aliquot of the sample with a known standard concentration of the antibiotic, isolating RNA from the aliquot of the sample, the RNA comprising a gene that shows a quantitatively different level of expression relative to the MIC of the antibiotic, reverse transcribing the RNA for the gene, amplifying a target of the gene to generate an amplified target, quantifying the amplified target to provide a quantified amplified target and to generate a value indicative of the MIC, and reporting the MIC as a result of the quantitative output for the gene.

Additional features and advantages of the embodiments of the invention will be set forth in the description which follows or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 shows a flexible pouch according to one embodiment of the present invention.

FIG. 2 shows an exploded perspective view of an instrument for use with the pouch of FIG. 1, including the pouch of FIG. 1, according to an example embodiment of the present invention.

FIG. 3 shows a partial cross-sectional view of the instrument of FIG. 2, including the bladder components of FIG. 2, with the pouch of FIG. 1 shown in dashed lines, according to an example embodiment of the present invention.

FIG. 4 shows a motor used in one illustrative embodiment of the instrument of FIG. 2.

FIG. 5A shows amplification curves for a generic antibiotic resistance gene lasI, where the Cp for the susceptible strain is earlier than the Cp for the resistant strain, regardless of whether the strain was incubated with an antibiotic. Four conditions are shown: Susceptible −ABX (-), Susceptible +ABX (- - -), Resistant −ABX (-^{●}-^{●●}-), Resistant +ABX (- - - -), wherein −ABX indicates no treatment with antibiotics and +ABX indicates treatment with antibiotics.

FIG. 5B shows amplification curves for a specific antibiotic resistance gene LexA, where the Cp for the susceptible strain is earlier than the Cp for the resistant strain only when the strain was incubated with an antibiotic. Four conditions are shown: Susceptible −ABX (-), Susceptible +ABX (- - -), Resistant −ABX (-^{●}-^{●●}-), Resistant +ABX (- - - -), wherein −ABX indicates no treatment with antibiotics and +ABX indicates treatment with antibiotics.

FIG. 6 shows Cp for the high copy target PA14_RS28865, when amplified in each of four conditions: −dsDNAse −RT, −dsDNAse +RT, +dsDNAse −RT, and +dsDNAse +RT.

FIGS. 7A-J show Cp for a number of different assays in the pouch of Example 2, in each of the following conditions: −dsDNAse −RT (left), +dsDNAse −RT (middle), and +dsDNAse +RT (right), wherein FIG. 7A is lexA, FIG. 7B is atpA, FIG. 7C is porin, FIG. 7D is oprD, FIG. 7E is RS25625, FIG. 7F is OmpA, FIG. 7G is yhbY, FIG. 7H is RS02955, FIG. 7I is rnpB, and FIG. 7J is PA14_RS28865.

FIG. 8 shows the Cp values for the lasI transcript in an illustrative pouch similar to FIG. 1.

FIGS. 9A and 9B present the relative expression level for the illustrative assay target lexA in both the resistant (FIG. 9A) and susceptible strain (FIG. 9B) when exposed to zero, 7.5, or 15 μg/mL ciprofloxacin at 10, 30 and 60 minutes of time.

FIG. 10 illustrates a block diagram of an exemplary embodiment of a thermal cycling system in accordance with aspects of the disclosure.

### DETAILED DESCRIPTION

Example embodiments are described below with reference to the accompanying drawings. Many different forms and embodiments are possible without deviating from the spirit and teachings of this disclosure and so the disclosure should not be construed as limited to the example embodiments set forth herein. Rather, these example embodiments are provided so that this disclosure will be thorough and complete, and will convey the scope of the disclosure to those skilled in the art. In the drawings, the sizes and relative sizes of layers and regions may be exaggerated for clarity. Like reference numbers refer to like elements throughout the description.

Unless defined otherwise, all terms (including technical and scientific terms) used herein have the same meaning as commonly understood by one of ordinary skill in the art to which the present disclosure pertains. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. While a number of methods and materials similar or equivalent to those described herein can be used in the practice of the present disclosure, only certain exemplary materials and methods are described herein.

All publications, patent applications, patents or other references mentioned herein are incorporated by reference in their entirety. In case of a conflict in terminology, the present specification is controlling.

Various aspects of the present disclosure, including devices, systems, methods, etc., may be illustrated with reference to one or more exemplary implementations. As used herein, the terms “exemplary” and “illustrative” mean “serving as an example, instance, or illustration,” and should not necessarily be construed as preferred or advantageous over other implementations disclosed herein. In addition, reference to an “implementation” or “embodiment” of the present disclosure or invention includes a specific reference to one or more embodiments thereof, and vice versa, and is intended to provide illustrative examples without limiting the scope of the invention, which is indicated by the appended claims rather than by the following description.

It will be noted that, as used in this specification and the appended claims, the singular forms “a,” “an,” and “the” include plural referents unless the content clearly dictates otherwise. Thus, for example, reference to “a tile” includes one, two, or more tiles. Similarly, reference to a plurality of referents should be interpreted as comprising a single referent and/or a plurality of referents unless the content and/or context clearly dictate otherwise. Thus, reference to “tiles” does not necessarily require a plurality of such tiles. Instead, it will be appreciated that independent of conjugation; one or more tiles are contemplated herein.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

As used herein, directional and/or arbitrary terms, such as “top,” “bottom,” “left,” “right,” “up,” “down,” “upper,” “lower,” “inner,” “outer,” “internal,” “external,” “interior,” “exterior,” “proximal,” “distal,” “forward,” “reverse,” and the like can be used solely to indicate relative directions and/or orientations and may not be otherwise intended to limit the scope of the disclosure, including the specification, invention, and/or claims.

It will be understood that when an element is referred to as being “coupled,” “connected,” or “responsive” to, or “on,” another element, it can be directly coupled, connected, or responsive to, or on, the other element, or intervening elements may also be present. In contrast, when an element is referred to as being “directly coupled,” “directly connected,” or “directly responsive” to, or “directly on,” another element, there are no intervening elements present.

Example embodiments of the present inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the present inventive concepts should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of example embodiments.

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By “sample” is meant an animal; a tissue or organ from an animal; a cell (either within a subject, taken directly from a subject, or a cell maintained in culture or from a cultured cell line); a cell lysate (or lysate fraction) or cell extract; a solution containing one or more molecules derived from a cell, cellular material, or viral material (e.g., a polypeptide or nucleic acid); or a solution containing a non-naturally occurring nucleic acid illustratively a cDNA or next-generation sequencing library, which is assayed as described herein. A sample may also be any body fluid or excretion (for example, but not limited to, blood, urine, stool, saliva, tears, bile, or cerebrospinal fluid) that may or may not contain host or pathogen cells, cell components, or nucleic acids. A sample may be treated, illustratively with an antibiotic, or may be used untreated.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), modified or treated bases and non-phosphodiester internucleoside linkages (e.g., peptide nucleic acid (PNA) or thiodiester linkages). In particular, nucleic acids can include, without limitation, DNA, cDNA, gDNA, ssDNA, dsDNA, RNA, including all RNA types such as miRNA, mtRNA, rRNA, including coding or non-coding regions, or any combination thereof.

By “probe,” “primer,” or “oligonucleotide” is meant a single-stranded nucleic acid molecule of defined sequence that can base-pair to a second nucleic acid molecule that contains a complementary sequence (the “target”). The stability of the resulting hybrid depends upon the length, GC content, and the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably-labeled, either radioactively labeled, fluorescently labeled, and/or non-radioactively labeled, by methods well-known to those skilled in the art. dsDNA binding dyes may be used to detect dsDNA. It is understood that a “primer” is specifically configured to be extended by a polymerase, whereas a “probe” or “oligonucleotide” may or may not be so configured. As a probe, the oligonucleotide could be used as part of many fluorescent PCR primer- and probe-based chemistries that are known in the art, including those sharing the use of fluorescence quenching and/or fluorescence resonance energy transfer (FRET) configurations, such as 5′ nuclease probes (TaqMan® probes), dual hybridization probes (HybProbes®), or Eclipse® probes or molecular beacons, or Amplifluor® assays, such as Scorpions®, LUX® or QZyme® PCR primers, including those with natural or modified bases.

By “dsDNA binding dyes” is meant dyes that fluoresce differentially when bound to double-stranded DNA than when bound to single-stranded DNA or free in solution, usually by fluorescing more strongly. While reference is made to dsDNA binding dyes, it is understood that any suitable dye may be used herein, with some non-limiting illustrative dyes described in U.S. Pat. No. 7,387,887, herein incorporated by reference. Other signal producing substances may be used for detecting nucleic acid amplification and melting, illustratively enzymes, antibodies, etc., as are known in the art.

By “specifically hybridizes” is meant that a probe, primer, or oligonucleotide recognizes and physically interacts (that is, base-pairs) with a substantially complementary nucleic acid (for example, a sample nucleic acid) under high stringency conditions, and does not substantially base pair with other nucleic acids.

By “high stringency conditions” is meant at about melting temperature (Tm) minus 5° C. (i.e., 5° below the Tm of the nucleic acid). Functionally, high stringency conditions are used to identify nucleic acid sequences having at least 80% sequence identity.

While PCR is the amplification method used in the examples herein, it is understood that any amplification method that uses a primer followed by a melting curve may be suitable. Such suitable procedures include polymerase chain reaction (PCR) of any type (single-step, two-steps, or others); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), next generation sequencing techniques, and the like. Therefore, when the term PCR is used, it should be understood to include other alternative amplification methods, including amino acid quantification methods. It is also understood that the methods included herein may be used for other biological and chemical processes that involve amplification that may be followed by melting curve analysis. For amplification methods without discrete cycles, reaction time may be used in lieu of measurements that are made in cycles or Cp, and additional reaction time may be added where additional PCR cycles are added in the embodiments described herein. It is understood that protocols may need to be adjusted accordingly.

When PCR and other biological and chemical processes that involve thermal cycling are used, it is understood that each cycle includes at least an annealing temperature and a denaturation temperature, wherein the denaturation phase involves heating to the denaturation temperature and the annealing phase involves cooling to the annealing temperature.

As used herein, “minimum inhibitory concentration” (“MIC”) is the lowest concentration of an antibiotic required to inhibit the growth of an organism.

As used herein, “breakpoint” is a concentration (often expressed as mg/L) of an antibiotic that defines whether a species of bacteria is susceptible or resistant to the antibiotic. If the MIC is less than or equal to the susceptibility breakpoint, the bacteria is considered to be susceptible to the antibiotic. If the MIC is greater than this value, the bacteria is considered to be resistant to the antibiotic. An intermediate group can also be reported, wherein the organism's MIC approaches or exceeds the threshold for normal antimicrobial dosing, but clinical response is possible with higher doses or if the antimicrobial concentrates at the site of infection.

While various examples herein reference human targets and human pathogens, these examples are illustrative only. Methods, kits, and devices described herein may be used to detect a wide variety of nucleic acid sequences from a wide variety of samples, including, human, veterinary, industrial, and environmental.

It is also understood that various implementations described herein can be used in combination with any other implementation described or disclosed, without departing from the scope of the present disclosure. Therefore, products, members, elements, devices, apparatus, systems, methods, processes, compositions, and/or kits according to certain implementations of the present disclosure can include, incorporate, or otherwise comprise properties, features, components, members, elements, steps, and/or the like described in other implementations (including systems, methods, apparatus, and/or the like) disclosed herein without departing from the scope of the present disclosure. Thus, reference to a specific feature in relation to one implementation should not be construed as being limited to applications only within said implementation.

The headings used herein are for organizational purposes only and are not meant to be used to limit the scope of the description or the claims. To facilitate understanding, like reference numerals have been used, where possible, to designate like elements common to the figures. Furthermore, where possible, like numbering of elements have been used in various figures. Furthermore, alternative configurations of a particular element may each include separate letters appended to the element number.

Various embodiments disclosed herein use a self-contained nucleic acid analysis pouch to assay a sample for the presence of various biological substances, illustratively antigens and nucleic acid sequences, illustratively in a single closed system. Such systems, including pouches and instruments for use with the pouches, are disclosed in more detail in U.S. Pat. Nos. 8,394,608; and 8,895,295; and U.S. Patent Application No. 2014-0283945, herein incorporated by reference. However, it is understood that such instruments and pouches are illustrative only, and the nucleic acid preparation and amplification reactions discussed herein may be performed in any of a variety of open or closed system sample vessels as are known in the art, including 96-well plates, plates of other configurations, arrays, carousels, and the like, using a variety of nucleic acid purification and amplification systems, as are known in the art. While the terms “sample well”, “amplification well”, “amplification container”, or the like are used herein, these terms are meant to encompass wells, tubes, and various other reaction containers, as are used in these amplification systems. Such amplification systems may include a single multiplex step in an amplification container and may optionally include a plurality of second-stage individual or lower-order multiplex reactions in a plurality of individual reaction wells. In one embodiment, the pouch is used to assay for multiple pathogens. The pouch may include one or more blisters used as sample wells, illustratively in a closed system. Illustratively, various steps may be performed in the optionally disposable pouch, including nucleic acid preparation, primary large volume multiplex PCR, dilution of primary amplification product, and secondary PCR, culminating with optional real-time detection or post-amplification analysis such as melting-curve analysis. Further, it is understood that while the various steps may be performed in pouches of the present invention, one or more of the steps may be omitted for certain uses, and the pouch configuration may be altered accordingly.

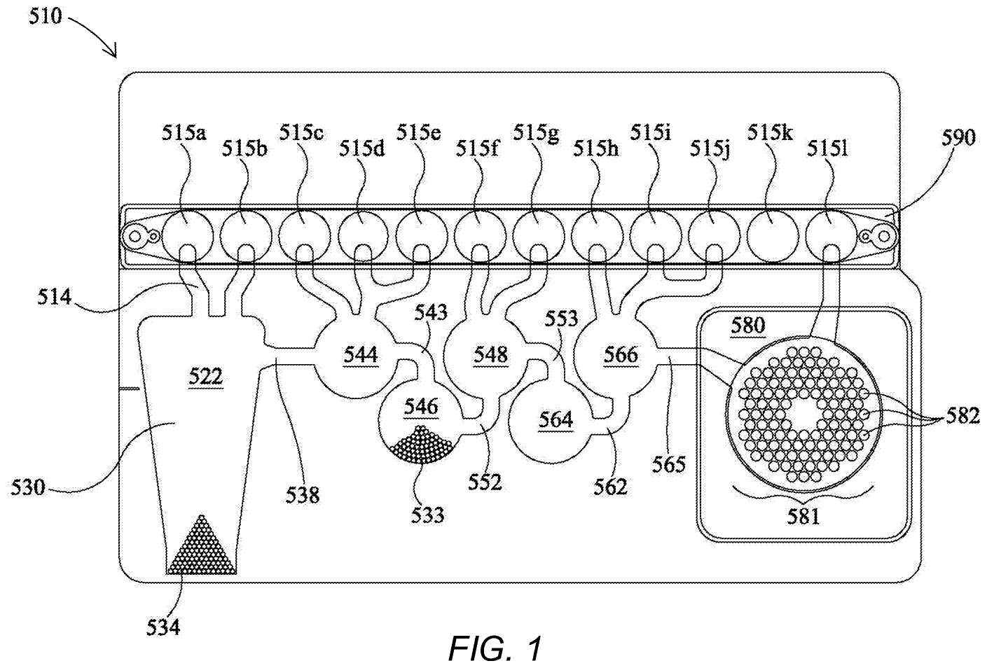

FIG. 1 shows an illustrative pouch 510 that may be used in various embodiments, or may be reconfigured for various embodiments. Pouch 51Q is similar to FIG. 15 of U.S. Pat. No. 8,895,295, with like items numbered the same. Fitment 590 is provided with entry channels 515a through 515l, which also serve as reagent reservoirs or waste reservoirs. Illustratively, reagents may be freeze dried in fitment 590 and rehydrated prior to use. Blisters 522, 544, 546, 548, 564, and 566, with their respective channels 514, 538, 543, 552, 553, 562, and 565 are similar to blisters of the same number of FIG. 15 of U.S. Pat. No. 8,895,295. Second-stage reaction zone 580 of FIG. 1 is similar to that of U.S. Pat. No. 8,895,295, but the second-stage wells 582 of high density array 581 are arranged in a somewhat different pattern. The more circular pattern of high density array 581 of FIG. 1 eliminates wells in corners and may result in more uniform filling of second-stage wells 582. As shown, the high density array 581 is provided with 102 second-stage wells 582. Pouch 510 is suitable for use in the FilmArray® instrument (BioFire Diagnostics, LLC, Salt Lake City, Utah). However, it is understood that the pouch embodiment is illustrative only.

While other containers may be used, illustratively, pouch 510 is formed of two layers of a flexible plastic film or other flexible material such as polyester, polyethylene terephthalate (PET), polycarbonate, polypropylene, polymethylmethacrylate, and mixtures thereof that can be made by any process known in the art, including extrusion, plasma deposition, and lamination. Metal foils or plastics with aluminum lamination also may be used. Other barrier materials are known in the art that can be sealed together to form the blisters and channels. If plastic film is used, the layers may be bonded together, illustratively by heat sealing. Illustratively, the material has low nucleic acid binding capacity.

For embodiments employing fluorescent monitoring, plastic films that are adequately low in absorbance and auto-fluorescence at the operative wavelengths are preferred. Such material could be identified by testing different plastics, different plasticizers, and composite ratios, as well as different thicknesses of the film. For plastics with aluminum or other foil lamination, the portion of the pouch that is to be read by a fluorescence detection device can be left without the foil. For example, if fluorescence is monitored in second-stage wells 582 of the second-stage reaction zone 580 of pouch 510, then one or both layers at wells 582 would be left without the foil. In the example of PCR, film laminates composed of polyester (Mylar, DuPont, Wilmington Del.) of about 0.0048 inch (0.1219 mm) thick and polypropylene films of 0.001-0.003 inch (0.025-0.076 mm) thick perform well. Illustratively, pouch 510 is made of a clear material capable of transmitting approximately 80%-90% of incident light.

In the illustrative embodiment, the materials are moved between blisters by the application of pressure, illustratively pneumatic pressure, upon the blisters and channels. Accordingly, in embodiments employing pressure, the pouch material illustratively is flexible enough to allow the pressure to have the desired effect. The term “flexible” is herein used to describe a physical characteristic of the material of pouch. The term “flexible” is herein defined as readily deformable by the levels of pressure used herein without cracking, breaking, crazing, or the like. For example, thin plastic sheets, such as Saran™ wrap and Ziploc® bags, as well as thin metal foil, such as aluminum foil, are flexible. However, only certain regions of the blisters and channels need be flexible, even in embodiments employing pneumatic pressure. Further, only one side of the blisters and channels need to be flexible, as long as the blisters and channels are readily deformable. Other regions of the pouch 51Q may be made of a rigid material or may be reinforced with a rigid material.

Illustratively, a plastic film is used for pouch 510. A sheet of metal, illustratively aluminum, or other suitable material, may be milled or otherwise cut, to create a die having a pattern of raised surfaces. When fitted into a pneumatic press (illustratively A-5302-PDS, Janesville Tool Inc., Milton Wis.), illustratively regulated at an operating temperature of 195° C., the pneumatic press works like a printing press, melting the sealing surfaces of plastic film only where the die contacts the film. Various components, such as PCR primers (illustratively spotted onto the film and dried), antigen binding substrates, magnetic beads, and zirconium silicate beads may be sealed inside various blisters as the pouch 510 is formed. Reagents for sample processing can be spotted onto the film prior to sealing, either collectively or separately. In one embodiment, nucleotide tri-phosphates (NTPs) are spotted onto the film separately from polymerase and primers, essentially eliminating activity of the polymerase until the reaction is hydrated by an aqueous sample. If the aqueous sample has been heated prior to hydration, this creates the conditions for a true hot-start PCR and reduces or eliminates the need for expensive chemical hot-start components.

Pouch 510 may be used in a manner similar to that described in U.S. Pat. No. 8,895,295. In one illustrative embodiment, a 300 μl mixture comprising the sample to be tested (100 μl) and lysis buffer (200 μl) is injected into an injection port (not shown) in fitment 590 near entry channel 515a, and the sample mixture is drawn into entry channel 515a. Water is also injected into a second injection port (not shown) of the fitment 590 adjacent entry channel 515l, and is distributed via a channel (not shown) provided in fitment 590, thereby hydrating up to eleven different reagents, each of which were previously provided in dry form at entry channels 515b through 515l. These reagents illustratively may include freeze-dried PCR reagents, DNA extraction reagents, wash solutions, immunoassay reagents, or other chemical entities. Illustratively, the reagents are for nucleic acid extraction, first-stage multiplex PCR, dilution of the multiplex reaction, and preparation of second-stage PCR reagents, as well as control reactions. In the embodiment shown in FIG. 1, all that need be injected is the sample solution in one injection port and water in the other injection port. After injection, the two injection ports may be sealed. For more information on various configurations of pouch 510 and fitment 590, see U.S. Pat. No. 8,895,295, already incorporated by reference.

After injection, the sample is moved from injection channel 515a to lysis blister 522 via channel 514. Lysis blister 522 is provided with beads or particles 534, such as ceramic beads, and is configured for vortexing via impaction using rotating blades or paddles provided within the FilmArray® instrument. Bead-milling, by shaking or vortexing the sample in the presence of lysing particles such as zirconium silicate (ZS) beads 534, is an effective method to form a lysate. It is understood that, as used herein, terms such as “lyse,” “lysing,” and “lysate” are not limited to rupturing cells, but that such terms include disruption of non-cellular particles, such as viruses.

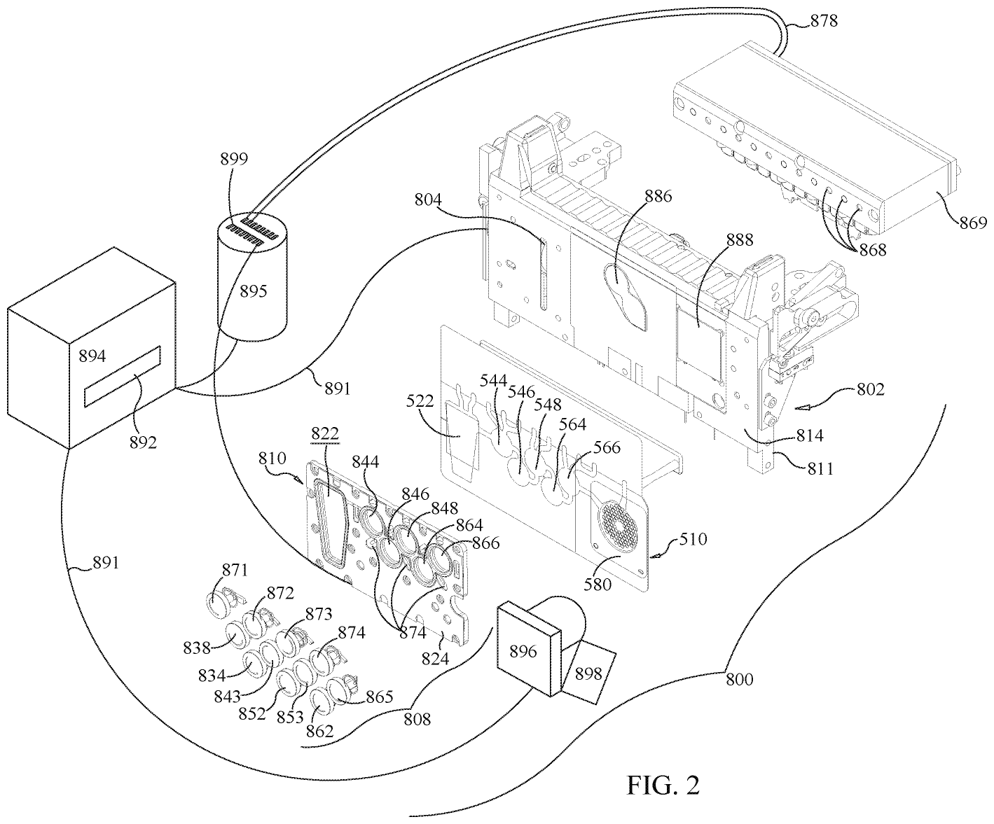

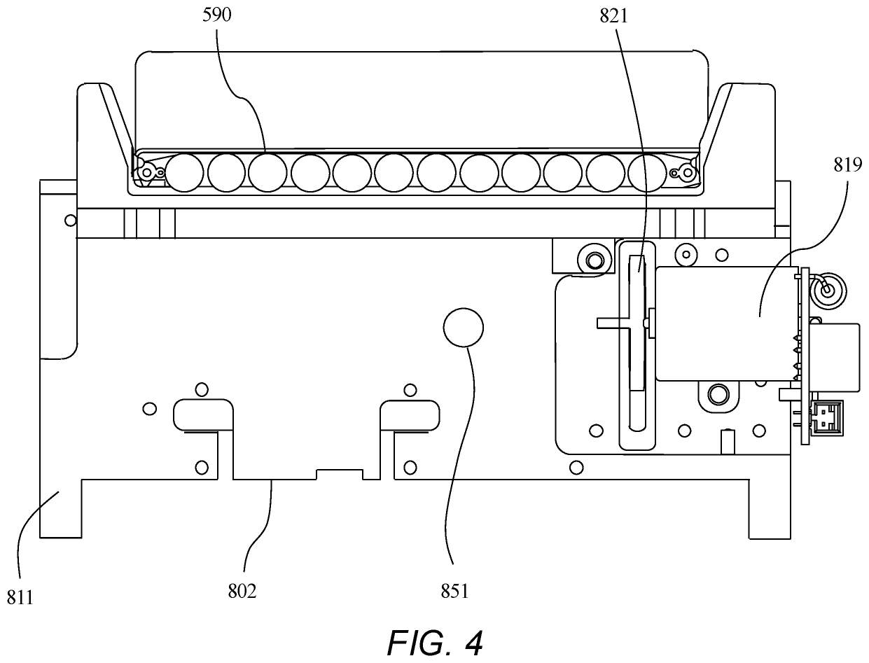

FIG. 4 shows a bead beating motor 819, comprising blades 821 that may be mounted on a first side 811 of support member 802, of instrument 800 shown in FIG. 2. Blades may extend through slot 804 to contact pouch 510. It is understood, however, that motor 819 may be mounted on other structures of instrument 800. In one illustrative embodiment, motor 819 is a Mabuchi RC-280SA-2865 DC Motor (Chiba, Japan), mounted on support member 802. In one illustrative embodiment, the motor is turned at 5,000 to 25,000 rpm, more illustratively 10,000 to 20,000 rpm, and still more illustratively approximately 15,000 to 18,000 rpm. For the Mabuchi motor, it has been found that 7.2V provides sufficient rpm for lysis. It is understood, however, that the actual speed may be somewhat slower when the blades 821 are impacting pouch 510. Other voltages and speeds may be used for lysis depending on the motor and paddles used. Optionally, controlled small volumes of air may be provided into the bladder 822 adjacent lysis blister 522. It has been found that in some embodiments, partially filling the adjacent bladder with one or more small volumes of air aids in positioning and supporting lysis blister during the lysis process. Alternatively, other structure, illustratively a rigid or compliant gasket or other retaining structure around lysis blister 522, can be used to restrain pouch 510 during lysis. It is also understood that motor 819 is illustrative only, and other devices may be used for milling, shaking, or vortexing the sample.

Once the cells have been adequately lysed, the sample is moved through channel 538, blister 544, and channel 543, to blister 546, where the sample is mixed with a nucleic acid-binding substance, such as silica-coated magnetic beads 533. The mixture is allowed to incubate for an appropriate length of time, illustratively approximately 10 seconds to 10 minutes. A retractable magnet located within the instrument adjacent blister 546 captures the magnetic beads 533 from the solution, forming a pellet against the interior surface of blister 546. The liquid is then moved out of blister 546 and back through blister 544 and into blister 522, which is now used as a waste receptacle. One or more wash buffers from one or more of injection channels 515c to 515e are provided via blister 544 and channel 543 to blister 546. Optionally, the magnet is retracted and the magnetic beads 533 are washed by moving the beads back and forth from blisters 544 and 546 via channel 543. Once the magnetic beads 533 are washed, the magnetic beads 533 are recaptured in blister 546 by activation of the magnet, and the wash solution is then moved to blister 522. This process may be repeated as necessary to wash the lysis buffer and sample debris from the nucleic acid-binding magnetic beads 533.

After washing, elution buffer stored at injection channel 515f is moved to blister 548, and the magnet is retracted. The solution is cycled between blisters 546 and 548 via channel 552, breaking up the pellet of magnetic beads 533 in blister 546 and allowing the captured nucleic acids to dissociate from the beads and come into solution. The magnet is once again activated, capturing the magnetic beads 533 in blister 546, and the eluted nucleic acid solution is moved into blister 548.

First-stage PCR master mix from injection channel 515g is mixed with the nucleic acid sample in blister 548. Optionally, the mixture is mixed by forcing the mixture between 548 and 564 via channel 553. After several cycles of mixing, the solution is contained in blister 564, where a pellet of first-stage PCR primers is provided, at least one set of primers for each target, and first-stage multiplex PCR is performed. If RNA targets are present, a reverse-transcription (RT) step using a suitable reverse-transcription enzyme may be performed prior to or simultaneously with the first-stage multiplex PCR. First-stage multiplex PCR temperature cycling in the FilmArray® instrument is illustratively performed for 15-30 cycles, although other levels of amplification may be desirable, depending on the requirements of the specific application. The first-stage PCR master mix may be any of various master mixes, as are known in the art. In one illustrative example, the first-stage PCR master mix may be any of the chemistries disclosed in US2015/0118715, herein incorporated by reference, for use with PCR protocols taking 20 seconds or less per cycle.

After first-stage PCR has proceeded for the desired number of cycles, the sample may be diluted, illustratively by forcing most of the sample back into blister 548, leaving only a small amount in blister 564, and adding second-stage PCR master mix from injection channel 515i. Alternatively, a dilution buffer from 515i may be moved to blister 566 then mixed with the amplified sample in blister 564 by moving the fluids back and forth between blisters 564 and 566. If desired, dilution may be repeated several times, using dilution buffer from injection channels 515j and 515k, or injection channel 515k may be reserved for sequencing or for other post-PCR analysis, and then adding second-stage PCR master mix from injection channel 515h to some or all of the diluted amplified sample. It is understood that the level of dilution may be adjusted by altering the number of dilution steps or by altering the percentage of the sample discarded prior to mixing with the dilution buffer or second-stage PCR master mix comprising components for amplification, illustratively a polymerase, dNTPs, and a suitable buffer, although other components may be suitable, particularly for non-PCR amplification methods. If desired, this mixture of the sample and second-stage PCR master mix may be pre-heated in blister 564 prior to movement to second-stage wells 582 for second-stage amplification. Such preheating may obviate the need for a hot-start component (antibody, chemical, or otherwise) in the second-stage PCR mixture.

The illustrative second-stage PCR master mix is incomplete, lacking primer pairs, and each of the 102 second-stage wells 582 is pre-loaded with a specific PCR primer pair (or sometimes multiple pairs of primers). If desired, second-stage PCR master mix may lack other reaction components, and these components may be pre-loaded in the second-stage wells 582 as well. Each primer pair may be similar to or identical to a first-stage PCR primer pair or may be nested within the first-stage primer pair. Movement of the sample from blister 564 to the second-stage wells 582 completes the PCR reaction mixture. Once high density array 581 is filled, the individual second-stage reactions are sealed in their respective second-stage blisters by any number of means, as is known in the art. Illustrative ways of filling and sealing the high density array 581 without cross-contamination are discussed in U.S. Pat. No. 8,895,295, already incorporated by reference. Illustratively, the various reactions in wells 582 of high density array 581 are simultaneously thermal cycled, illustratively with one or more Peltier devices, although other means for thermal cycling are known in the art.

In certain embodiments, second-stage PCR master mix contains the dsDNA binding dye LCGreen® Plus (BioFire Diagnostics, LLC) to generate a signal indicative of amplification. However, it is understood that this dye is illustrative only, and that other signals may be used, including other dsDNA binding dyes and probes that are labeled fluorescently, radioactively, chemiluminescently, enzymatically, or the like, as are known in the art. Alternatively, wells 582 of array 581 may be provided without a signal, with results reported through subsequent processing.

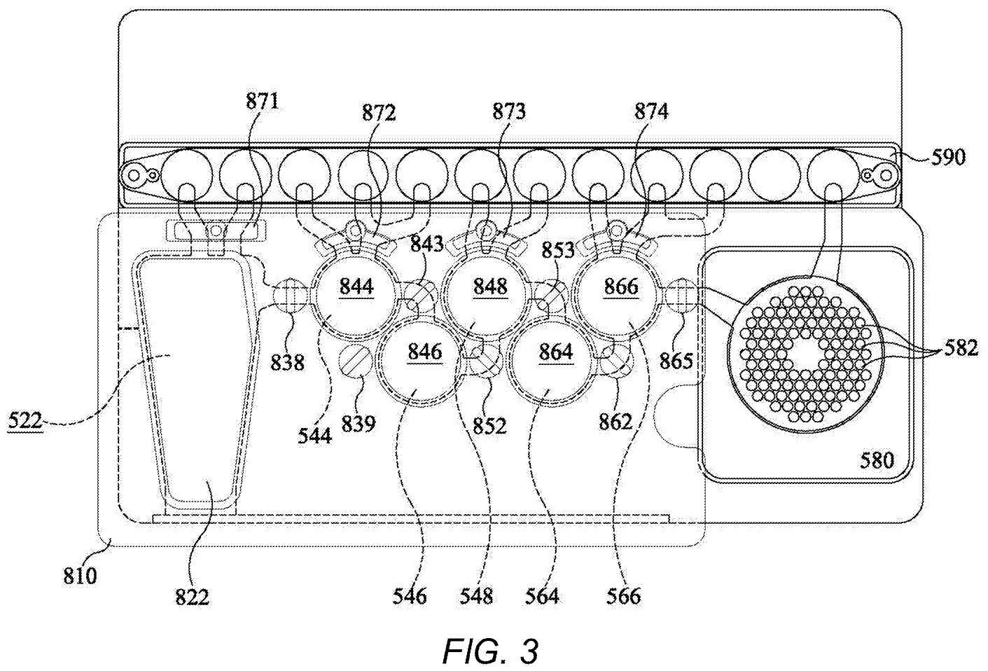

When pneumatic pressure is used to move materials within pouch 510, in one embodiment a “bladder” may be employed. The bladder assembly 810, a portion of which is shown in FIGS. 2 and 3, includes a bladder plate 824 housing a plurality of inflatable bladders 822, 844, 846, 848, 864, and 866, each of which may be individually inflatable, illustratively by a compressed gas source. Because the bladder assembly 810 may be subjected to compressed gas and used multiple times, the bladder assembly 810 may be made from tougher or thicker material than the pouch. Alternatively, bladders 822, 844, 846, 848, 864, and 866 may be formed from a series of plates fastened together with gaskets, seals, valves, and pistons. Other arrangements are within the scope of this invention.

Success of the secondary PCR reactions is dependent upon template generated by the multiplex first-stage reaction. Typically, PCR is performed using DNA of high purity. Methods such as phenol extraction or commercial DNA extraction kits provide DNA of high purity. Samples processed through the pouch 510 may require accommodations be made to compensate for a less pure preparation. PCR may be inhibited by components of biological samples, which is a potential obstacle. Illustratively, hot-start PCR, higher concentration of taq polymerase enzyme, adjustments in MgCl₂ concentration, adjustments in primer concentration, and addition of adjuvants (such as DMSO, TMSO, or glycerol) optionally may be used to compensate for lower nucleic acid purity. While purity issues are likely to be more of a concern with first-stage amplification and single-stage PCR, it is understood that similar adjustments may be provided in the second-stage amplification as well.

When pouch 510 is placed within the instrument 800, the bladder assembly 810 is pressed against one face of the pouch 510, so that if a particular bladder is inflated, the pressure will force the liquid out of the corresponding blister in the pouch 510. In addition to bladders corresponding to many of the blisters of pouch 510, the bladder assembly 810 may have additional pneumatic actuators, such as bladders or pneumatically-driven pistons, corresponding to various channels of pouch 510. FIGS. 2 and 3 show an illustrative plurality of pistons or hard seals 838, 843, 852, 853, and 865 that correspond to channels 538, 543, 553, and 565 of pouch 510, as well as seals 871, 872, 873, 874 that minimize backflow into fitment 590. When activated, hard seals 838, 843, 852, 853, and 865 form pinch valves to pinch off and close the corresponding channels. To confine liquid within a particular blister of pouch 510, the hard seals are activated over the channels leading to and from the blister, such that the actuators function as pinch valves to pinch the channels shut. Illustratively, to mix two volumes of liquid in different blisters, the pinch valve actuator sealing the connecting channel is activated, and the pneumatic bladders over the blisters are alternately pressurized, forcing the liquid back and forth through the channel connecting the blisters to mix the liquid therein. The pinch valve actuators may be of various shapes and sizes and may be configured to pinch off more than one channel at a time. While pneumatic actuators are discussed herein, it is understood that other ways of providing pressure to the pouch are contemplated, including various electromechanical actuators such as linear stepper motors, motor-driven cams, rigid paddles driven by pneumatic, hydraulic or electromagnetic forces, rollers, rocker-arms, and in some cases, cocked springs. In addition, there are a variety of methods of reversibly or irreversibly closing channels in addition to applying pressure normal to the axis of the channel. These include kinking the bag across the channel, heat-sealing, rolling an actuator, and a variety of physical valves sealed into the channel such as butterfly valves and ball valves. Additionally, small Peltier devices or other temperature regulators may be placed adjacent the channels and set at a temperature sufficient to freeze the fluid, effectively forming a seal. Also, while the design of FIG. 1 is adapted for an automated instrument featuring actuator elements positioned over each of the blisters and channels, it is also contemplated that the actuators could remain stationary, and the pouch 510 could be transitioned in one or two dimensions such that a small number of actuators could be used for several of the processing stations including sample disruption, nucleic-acid capture, first and second-stage PCR, and other applications of the pouch 510 such as immuno-assay and immuno-PCR. Rollers acting on channels and blisters could prove particularly useful in a configuration in which the pouch 510 is translated between stations. Thus, while pneumatic actuators are used in the presently disclosed embodiments, when the term “pneumatic actuator” is used herein, it is understood that other actuators and other ways of providing pressure may be used, depending on the configuration of the pouch and the instrument.

Other prior art instruments teach PCR within a sealed flexible container. See, e.g., U.S. Pat. Nos. 6,645,758 and 6,780,617, and 9,586,208, herein incorporated by reference. However, including the cell lysis within the sealed PCR vessel can improve ease of use and safety, particularly if the sample to be tested may contain a biohazard. In the embodiments illustrated herein, the waste from cell lysis, as well as that from all other steps, remains within the sealed pouch. However, it is understood that the pouch contents could be removed for further testing.

FIG. 2 shows an illustrative instrument 800 that could be used with pouch 510. Instrument 800 includes a support member 802 that could form a wall of a casing or be mounted within a casing. Instrument 800 may also include a second support member (not shown) that is optionally movable with respect to support member 802, to allow insertion and withdrawal of pouch 510. Illustratively, a lid may cover pouch 510 once pouch 510 has been inserted into instrument 800. In another embodiment, both support members may be fixed, with pouch 510 held into place by other mechanical means or by pneumatic pressure.

In the illustrative example, heaters 886 and 888 are mounted on support member 802. However, it is understood that this arrangement is illustrative only and that other arrangements are possible. Bladder plate 810, with bladders 822, 844, 846, 848, 864, 866, hard seals 838, 843, 852, 853, seals 871, 872, 873, 874 form bladder assembly 808 may illustratively be mounted on a moveable support structure that may be moved toward pouch 510, such that the pneumatic actuators are placed in contact with pouch 510. When pouch 510 is inserted into instrument 800 and the movable support member is moved toward support member 802, the various blisters of pouch 510 are in a position adjacent to the various bladders of bladder assembly 810 and the various seals of assembly 808, such that activation of the pneumatic actuators may force liquid from one or more of the blisters of pouch 510 or may form pinch valves with one or more channels of pouch 510. The relationship between the blisters and channels of pouch 510 and the bladders and seals of assembly 808 is illustrated in more detail in FIG. 3.

Each pneumatic actuator is connected to compressed air source 895 via valves 899. While only several hoses 878 are shown in FIG. 2, it is understood that each pneumatic fitting is connected via a hose 878 to the compressed gas source 895. Compressed gas source 895 may be a compressor, or, alternatively, compressed gas source 895 may be a compressed gas cylinder, such as a carbon dioxide cylinder. Compressed gas cylinders are particularly useful if portability is desired. Other sources of compressed gas are within the scope of this invention.

Assembly 808 is illustratively mounted on a movable support member, although it is understood that other configurations are possible.

Several other components of instrument 81Q are also connected to compressed gas source 895. A magnet 850, which is mounted on a second side 814 of support member 802, is illustratively deployed and retracted using gas from compressed gas source 895 via hose 878, although other methods of moving magnet 850 are known in the art. Magnet 850 sits in recess 851 in support member 802. It is understood that recess 851 can be a passageway through support member 802, so that magnet 850 can contact blister 546 of pouch 510. However, depending on the material of support member 802, it is understood that recess 851 need not extend all the way through support member 802, as long as when magnet 850 is deployed, magnet 850 is close enough to provide a sufficient magnetic field at blister 546, and when magnet 850 is retracted, magnet 850 does not significantly affect any magnetic beads 533 present in blister 546. While reference is made to retracting magnet 850, it is understood that an electromagnet may be used and the electromagnet may be activated and inactivated by controlling flow of electricity through the electromagnet. Thus, while this specification discusses withdrawing or retracting the magnet, it is understood that these terms are broad enough to incorporate other ways of withdrawing the magnetic field. It is understood that the pneumatic connections may be pneumatic hoses or pneumatic air manifolds, thus reducing the number of hoses or valves required.

The various pneumatic pistons 868 of pneumatic piston array 869 are also connected to compressed gas source 895 via hoses 878. While only two hoses 878 are shown connecting pneumatic pistons 868 to compressed gas source 895, it is understood that each of the pneumatic pistons 868 are connected to compressed gas source 895. Twelve pneumatic pistons 868 are shown.

A pair of heating/cooling devices, illustratively Peltier heaters, are mounted on a second side 814 of support 802. First-stage heater 886 is positioned to heat and cool the contents of blister 564 for first-stage PCR. Second-stage heater 888 is positioned to heat and cool the contents of second-stage blisters 582 of pouch 510, for second-stage PCR. It is understood, however, that these heaters could also be used for other heating purposes, and that other heaters may be use, as appropriate for the particular application. Other configurations are possible.

When fluorescent detection is desired, an optical array 890 may be provided. As shown in FIG. 2, optical array 890 includes a light source 898, illustratively a filtered LED light source, filtered white light, or laser illumination, and a camera 896. Camera 896 illustratively has a plurality of photodetectors each corresponding to a second-stage well 582 in pouch 510. Alternatively, camera 896 may take images that contain all of the second-stage wells 582, and the image may be divided into separate fields corresponding to each of the second-stage wells 582. Depending on the configuration, optical array 890 may be stationary, or optical array 890 may be placed on movers attached to one or more motors and moved to obtain signals from each individual second-stage well 582. It is understood that other arrangements are possible.

As shown, a computer 894 controls valves 899 of compressed air source 895, and thus controls all of the pneumatics of instrument 800. Computer 894 also controls heaters 886 and 888, and optical array 890. Each of these components is connected electrically, illustratively via cables 891, although other physical or wireless connections are within the scope of this invention. It is understood that computer 894 may be housed within instrument 800 or may be external to instrument 800. Further, computer 894 may include built-in circuit boards that control some or all of the components, may calculate amplification curves, melting curves, Cps, differences between Cps (ΔCp) for different wells (or absolute values of the difference between Cps), standard curves, and other related data, and may also include an external computer, such as a desktop or laptop PC, to receive and display data from the optical array. An interface, illustratively a keyboard interface, may be provided including keys for inputting information and variables such as temperatures, cycle times, etc. Illustratively, a display 892 is also provided. Display 892 may be an LED, LCD, or other such display, for example.

Antibiotic susceptibility can be measured on a molecular level by detecting transcriptional differences in susceptible and resistant bacteria in response to antibiotic exposure.

By measuring transcriptional differences, a high positive predictive value (“PPV”), true positives/(true positives+false positives), is desirable. With this information, a physician can change therapy, including antibiotic escalation, de-escalation, or a change to a different antibiotic.

A negative predictive value (“NPV”), true negatives/(true negatives+false negatives) is currently more difficult to interpret. An NPV does not tell you whether the organism is sensitive, as with current understanding, there are too many resistance mechanisms to have any sort of reasonable NPV. Thus, in some embodiments, NPV may not be as useful.

A susceptible bacterium that is treated with a sufficient dose of an antibiotic will eventually die. However, prior to the bacterium showing a phenotypic trait that can be detected with microbiologic test, the bacterium undergoes biochemical changes that should be detectable with a molecular test. One such test is transcriptome remodeling. The following example is focused on identifying transcriptome differences that distinguish and predict the death upon exposure to an antibiotic.

### Example 1

Antibiotic susceptibility can be measured on a molecular level by detecting transcriptional differences in susceptible and resistant bacteria in response to antibiotic exposure. These transcriptional differences can be discovered illustratively using RNA sequencing or cDNA microarray analysis. The large multiplex and reverse-transcription capabilities of multiplex systems such as the FilmArray System, as described above, could facilitate measuring antibiotic susceptibility for multiple bacteria and antibiotics. In this example, specific and generic bacteria-antibiotic combinations are targeted after exposure to an antibiotic to determine if differences can be detected between susceptible and resistant strains. It is understood that such methods could be extrapolated to other antibiotics or mixtures of antibiotics.

In prior art methods, both susceptible and resistant cultures are grown to early log phase and are then exposed to antibiotic or no antibiotic (control) at breakpoint, illustratively for 30 minutes although other times can be used. The cells are then harvested and prepared for RNA sequencing. In such methods, a large quantity of computer power is needed to try to understand the differences in mRNA expression between susceptible and resistant strains. Illustratively, to make sense of the data from such prior art methods, the data would need to be cleaned to obtain good quality reads, the reads would need to be normalized to compare equal sampling, the transcripts would need to be quantified, and the same transcripts would need to be compared between the four conditions (two strains (susceptible or resistant), each +/− antibiotic, provides four test conditions). The mRNAs that provide the most difference between the conditions would then be identified. mRNAs that do not differentiate between the conditions may be used as an internal reference for normalization between samples. A prior study (Barczak) identified four markers with differential transcriptional responses to Ciprofloxacin (CIP) in susceptible vs. resistant strains.

An initial study for a multiplex PCR-based detection uses:

P. aeruginosa, two strains: one that is resistant and the other susceptible.
S1=susceptible to Ciprofloxacin
R1=resistant to Ciprofloxacin

The antibiotic Ciprofloxacin, for each of the two strains.

A no antibiotic control for each of the two strains.

In this example, each strain (S1 and R1) was grown to 0.5 OD₆₀₀ (-1×10⁸ CFU/mL) and each was treated with or without 15 μg/mL of Ciprofloxacin for 10 minutes (two strains, each +/− antibiotic, provides four test conditions). It is understood that the OD and antibiotic incubation time are illustrative only and that other concentrations and times may be used. cDNA was generated by extracting on Magnapure using TNA kit, following bacterial lysis protocol, quantifying using Qubit RNA HS Assay Kit (Q32852), genomic DNA removed and cDNA generated using Maxima H Minus cDNA Kit with dsDNAse (M1682).

Four reference genes, expression for each of which is expected to remain relatively constant between susceptible and resistant strains, and remain constant in the presence or absence of antibiotic, were used. The four references genes are proC, rpoD, piv, and pcaH. In benchtop experiments, all four of these genes provided similar Cps for each of four test conditions, that is for each gene similar Cps were obtained for susceptible and resistant strains, each with and without antibiotic treatment. Thus, these four genes are appropriate reference genes and can be used to normalize results from other genes, illustratively due to differences in the number of cells/sample. In one illustrative embodiment, Cp for a reference gene may be used to normalize Cp across samples for one or more genes indicative of antibiotic resistance. While one reference gene may be used for this purpose, using a combination of reference genes may help reduce noise or erroneous results. Illustratively, a geometric mean of Cp of multiple reference genes may be used, although other methods of using a combination of genes are known in the art, Thus, one or all of these or other reference genes may be used to normalize Cp across samples in any of the embodiments herein. Further, while it may be helpful to have a bacterial load that is close to optimal for the system, because of this normalization, in various embodiments quantification may not require knowing the exact bacterial load.

Other genes show different patterns of expression between susceptible and resistant strains. Several of these genes are in the quorum sensing pathway or the iron uptake pathway, while the pathway for several others are unknown. Table 1 shows results obtained from benchtop experiments in testing the following twenty gene targets in the presence of antibiotics: lexA, sulA, recN, recA, prtN, ptrB, yhbY, LasI, RhlI, pqsH, pvdE, tonB, pvdA, ABC, PepSY, speD, PA14_RS20905, PA14_RS07980, PA14_RS07985, and coA.

TABLE 1

| Gene | Pathway | Sus/Res ΔCp |
|---|---|---|
| lexA | SOS | −2.2 |
| sulA | SOS | −4.7 |
| recN | SOS | <1 |
| recA | SOS | <1 |
| prtN | SOS | <1 |
| ptrB | SOS | 1.3 |
| yhbY | RNA binding | <1 |
| lasI | Quorum Sensing | −5.5 |
| rhlI | Quorum Sensing | −4.3 |
| pqsH | Quorum Sensing | −5.5 |
| pvdE | Iron Uptake | −7.8 |
| tonB | Iron Uptake | — |
| pvdA | Iron Uptake | — |
| ABC | Iron Uptake | — |
| pepSY | Nutrient Uptake | −9.8 |
| speD | — | 3.1 |
| PA14_RS20905 | Unknown | −11.3 |
| PA14_RS07980 | Unknown | −1.6 |
| PA14_RS07985 | Unknown | −1.7 |
| coA | — | −7.1 |

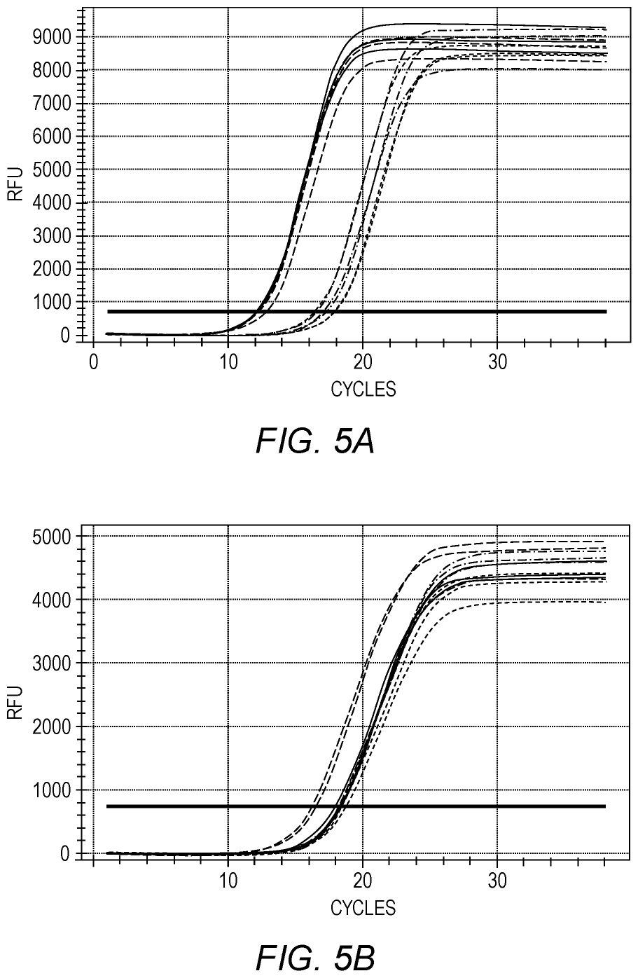

For some of these genes, a similar ΔCp between susceptible and resistant strains was found both with and without the presence of antibiotics. Such genes are referred to as “generic antibiotic resistance genes” as they distinguish between susceptible and resistant strains even in the absence of antibiotics. Several of these generic antibiotic resistance genes are in the quorum sensing pathway or the iron uptake pathway, while the pathway for several others are unknown. These genes included LasI, RhlI, pqsH, pvdE, PepSY, speD, PA14_RS20905, and coA. The amplification curves for LasI are shown in FIG. 5A. As shown in FIG. 5A for LasI, no antibiotics (“ABX”) are needed to see a gene expression difference between susceptible and resistant strains. The Cps for the amplification curves shown in FIG. 5A are as follows:

TABLE 2

| | | |
|---|---|---|
| cDNA | Susceptible −ABX | 10.44 |
| 1:100 | Susceptible +ABX | 10.55 |
| dilution | Resistant −ABX | 15.51 |
| | Resistant +ABX | 15.15 |

In the testing conditions used, for susceptible strains, the Cp is about 10.5, regardless of whether antibiotic is present, while the Cp for resistant strains is about 15, regardless of whether antibiotic is present. While most of these generic antibiotic resistance genes show an up-regulation in the susceptible strain, it is noted that speD showed down-regulation.

For other genes, significant ΔCp was found only in the presence of antibiotics. These “specific antibiotic resistance genes” include lexA, sulA, ptrB, and, PA14_RS07985, with ptrB showing up-regulation in the resistant strain. The amplification curves for lexA are shown in FIG. 5B, where the susceptible strain with antibiotic has a Cp about 2 cycles earlier than the other three conditions. For the other three conditions, the resistant strain with and without antibiotics had essentially the same Cp as the susceptible strain without antibiotics. The Cps are as follows:

TABLE 3

| | | |
|---|---|---|
| cDNA | Susceptible −ABX | 18.18 |
| 1:100 | Susceptible +ABX | 16.36 |
| dilution | Resistant −ABX | 18.39 |
| | Resistant +ABX | 18.55 |

It is expected that some combination of generic antibiotic resistance genes and/or specific antibiotic resistance genes can be used in a molecular test to determine whether an unknown sample is susceptible or resistant to antibiotics. The sample may be incubated with one or more antibiotics prior to testing, to test for both generic antibiotic resistance genes and specific antibiotic resistance genes.

It is noted above that some genes may be up-regulated, while other genes are down-regulated. Both may be used in a test for antibiotic resistance. Where multiple genes are used, in one illustrative example, the absolute value of the shift for each gene may be used to output a single value indicative of susceptibility or resistance. In another embodiment, a mathematical output coding for the resistant or susceptible phenotype of the bacterium is, for example, a linear combination of the real values (as opposed to the absolute values) or a polynomial combination of higher degree of real values of the delta Cps. Other methods for combining the shifts in Cp are known and may be used to generate a quantitative or semi-quantitative output.

The remaining genes tested showed a ΔCp of <1, and these genes were not chosen for further studies. While these genes were not studied further, it is noted that some or all of these other genes that do not show a significant difference between susceptible and resistant strains could be used as reference genes.

### Example 2

The above benchtop multiplex experiments demonstrate the feasibility of using a measure of cellular RNA concentration of generic antibiotic resistance genes and/or specific antibiotic resistance genes as a test for antibiotic resistance. The ability to measure the concentration of or detect the presence of a bacterial RNA transcript is hindered by the fact that a large number of these transcripts are present at a concentration of much less than one transcript per cell (Bartholomaus, et al.). The practical consequence of this is that the concentration (copies/cell) of cellular genomic DNA (at least 1 copy/cell) often far surpasses that of any cellular RNA transcript (often <<1 copy/cell). Because bacterial transcripts are usually identical in sequence to their genomic copy (across the open reading frame), the total signal in a multiplex RT-PCR based detection strategy represents DNA+RNA (where often [DNA]>[RNA]). Under these conditions, removing genomic DNA would be helpful in facilitating detection of the RNA signal.

DNA removal may be accomplished using a number of strategies, illustratively, by modification of cellular lysis conditions to enable selective release of RNA, modification of nucleic acid purification to select for RNA, selective removal of DNA from purified nucleic acids, and/or other methods as are known in the art. In one non-limiting example, the selective removal of DNA from an RNA+DNA mixture may be accomplished enzymatically by selection of a DNase enzyme with appropriate properties, such as being low in or essentially free from RNAse activity. In some embodiments, having high activity against duplex DNA or low activity against DNA/RNA hybrids (such as primer RNA binding) may be desirable. It has been found that DNAse activity for various commercial dsDNAses plateaus after generating fragments of a few hundred base pairs, sometimes even after lengthy incubations. While many DNAses are known, a few non-limiting examples include dsDNAse (Pandalus borealis, Recombinant, Engineered recombinant), DNase I (Bovine spleen, Recombinant, other sources), Par_DSN (Kamchatka crab), and DNase II, (Procine/Bovine spleen, Recombinant, other sources).

Illustratively, using dsDNAse from Pandalus borealis, substantial digestion is seen in 1-10 minutes, with plateau in 20-30 minutes. For a fast DNAse treatment, illustratively no more than 20 minutes, and more illustratively no more than 10 minutes, and perhaps 5 minutes or shorter, although other times are possible depending on the system and enzyme used, effective results are seen with amplicons of at least 300 bp, perhaps 500 bp or more, where possible, as such longer amplicon lengths are more likely to have at least one double-stranded cut in the DNA counterpart sequence, which would essentially prevent such DNA from being amplified. It is understood that this is illustrative only, and other amplicon lengths may be used, and also that temperature may also be used to control the speed of the DNAse reaction. Using longer amplicon lengths is counterintuitive in some situations, especially when fast assays are desirable, as longer amplicon lengths can require longer extension time. However, this can be partially offset by shorter DNAse times. In some assays, shorter amplicon lengths may be desired, or may even be necessary, illustratively due to shorter RNA starting material or desired primer binding sites.

In this illustrative example, a pouch similar to pouch 510 was developed to include a DNase treatment step by including DNAse, illustratively a dsDNAse from Pandalus borealis, freeze dried into the fitment, as well as a slight modification in the elution buffer that is freeze dried into injection channel 515e, although it is understood that this dsDNAse is illustrative only and that other DNAses can be used, as well as other DNA removal methods. For the DNAse step, subsequent to elution, the temperature is raised to a temperature suitable for the DNAse enzyme, illustratively 42° C., followed by reverse-transcription and first-stage multiplex PCR. In this example, the pouch contained 45 different assays of interest (including control genes such as rpoD), targets where mRNA is present in concentrations greater than genomic (“high copy targets”) such as PA14_RS28865, as well as a number of antibiotic resistance gene targets including recA (specific) and lasI (generic) that are present in concentrations lower than genomic. However, it is understood that this panel of assays is illustrative only, and any combination of assays may be used. In one illustrative embodiment, all assays are for RNA targets, illustratively mRNA targets, although other RNA targets may be suitable for detection and/or quantification using the methods provided herein. In this example, four similar pouches were developed, with and without the dsDNAse enzyme and elution buffer (+dsDNAse or −dsDNAse), each with and without a reverse-transcription enzyme (+RT or −RT).

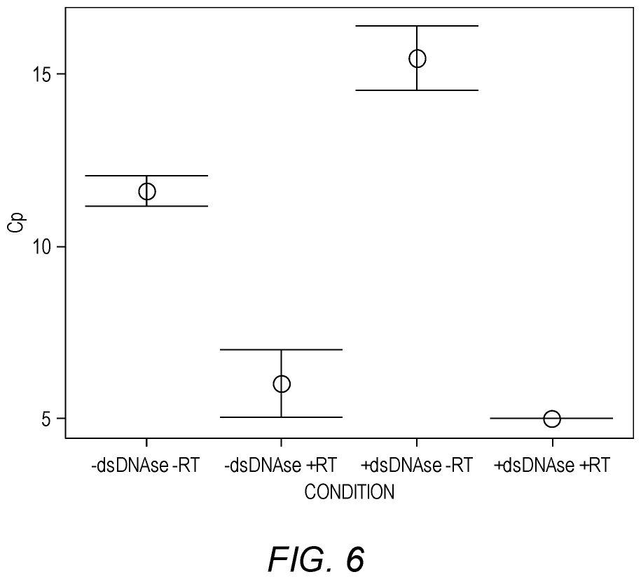

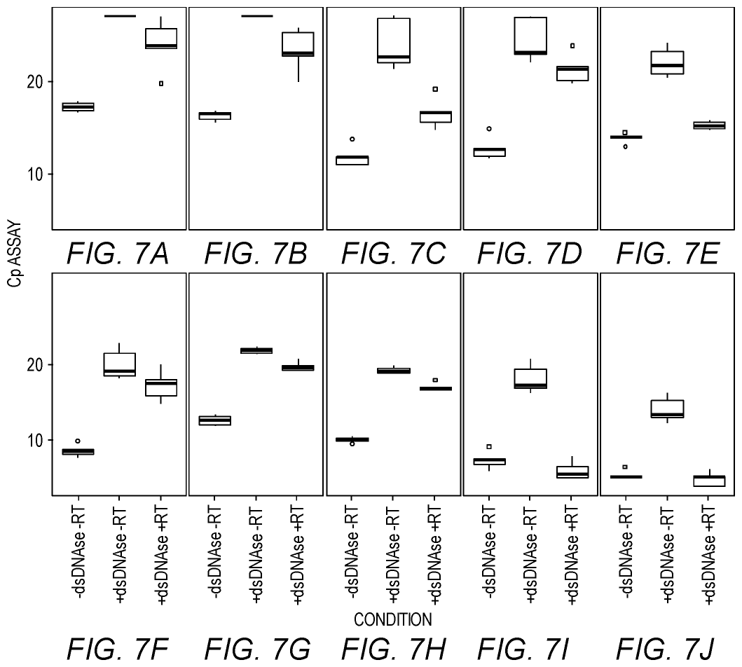

As expected, initial testing with this panel demonstrated that for the high copy target PA14_RS28865, an RNA-dependent signal was observed independent of dsDNAse treatment. As seen in FIG. 6, for this high copy target, reverse-transcription alone (without a dsDNAse step, −dsDNAse +RT) is sufficient to generate cDNA concentrations that detectably exceed genomic DNA concentrations. As expected for this high copy target, the Cp for the +dsDNAse +RT condition is generally equivalent to the Cp from the −dsDNAse +RT condition, indicating that the RNA concentration is unaffected by the dsDNAse treatment. It is noted that an earlier Cp is expected for targets that are provided in higher concentrations, so a lower value in FIGS. 6-7 indicates a higher concentration. FIGS. 7A-7J show the Cp for various other targets without DNAse treatment (−dsDNAse −RT), with DNAse treatment (+dsDNAse −RT), and with DNAse treatment followed by an RT step (+dsDNAse +RT). In contrast to the high copy target PA14_RS28865 (FIG. 7J), dsDNAse is desirable to detect an RNA-dependent signal. This is expected for targets with [RNA]<[DNA]. For lexA, atpA, oprD, RS25625, ompA, yhbY, RS02955, and rnpB (FIGS. 7A, 7B, 7D, 7E, 7F, 7G, 7H, and 7I) the Cp is observed to decrease when comparing the +dsDNAse −RT with the +dsDNAse +RT condition (middle and right boxes), indicating an increase in amplification and starting concentration. It is noted that in each case, the RNA-dependent Cp observed for these targets (+dsDNAse +RT, right) is greater than the Cp observed for the DNA-only signal (−dsDNAse −RT, left), confirming that the [RNA] is less than the [DNA]. It is believed that increasing reverse-transcription efficiency or dsDNAse efficiency would act to further differentiate the +dsDNAse +RT signal from the +dsDNAse −RT condition. These data demonstrate that for targets with [RNA]<[DNA], treatment with dsDNAse may be used to reduce the concentration of DNA to a level less than the RNA concentration. Other strategies that reduce DNA or selectively detect RNA may be used as well. Under these conditions, the observed Cp may be used to provide a measure of the concentration of RNA for the given assay target within the bacterial population introduced into the pouch. Early testing with Ciprofloxacin treatment, prior to injecting the sample into the pouch, resulted in expected changes in Cp for the susceptible antibiotic resistance genes.

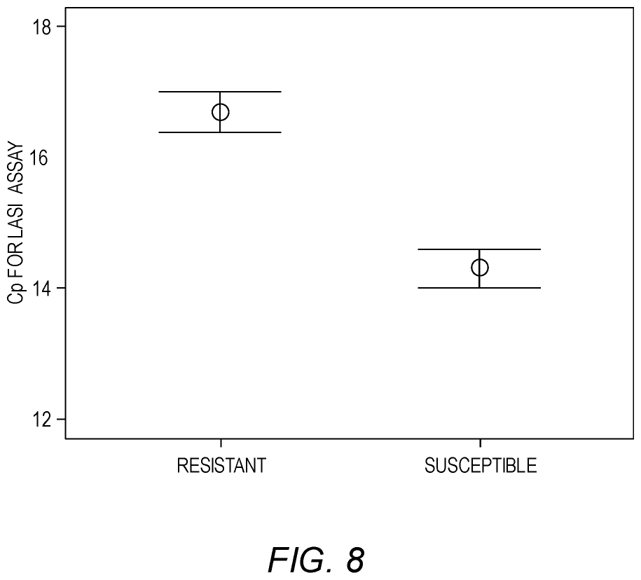

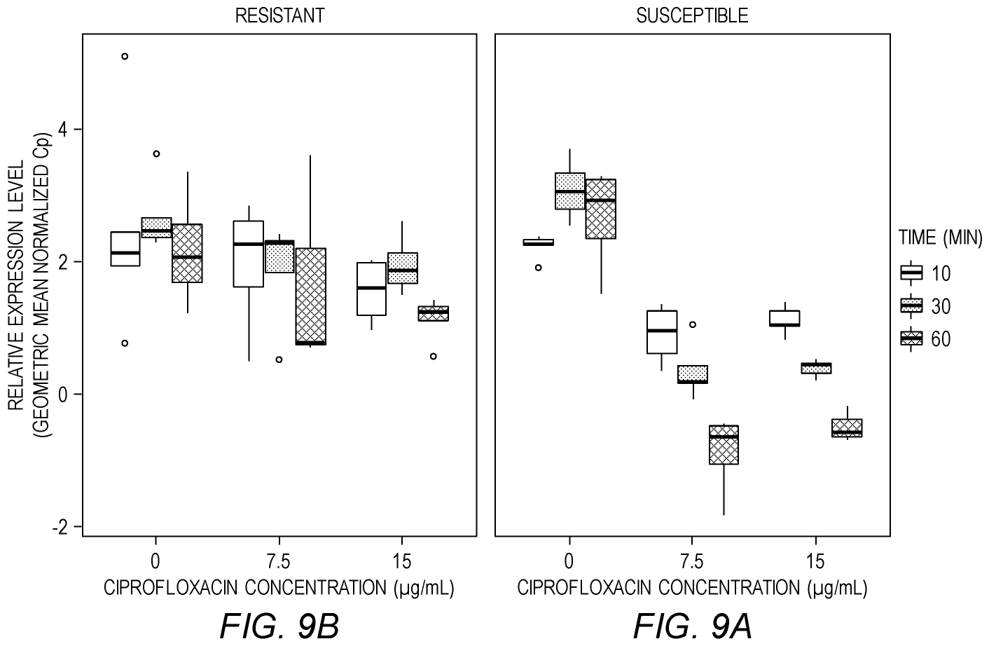

The ability of the illustrative pouch to function as a rapid phenotypic susceptibility test is demonstrated in FIGS. 8, 9A and 9B. As noted previously, generic antibiotic resistance genes may be used to discriminate susceptible from resistant strains by virtue of the fact that they are expressed at different levels in the two strains (without regard to the presence of an antibiotic). FIG. 8 shows the Cp values for the lasI transcript from this illustrative pouch (points show mean Cp (N=5) error bars are +\−sd). These data recapitulate the data obtained in bench testing that also show a higher expression of the lasI transcript in the susceptible strain (see Table 2 above).

As previously noted, specific antibiotic resistance genes enable discrimination of susceptible from resistant strains by detecting changes in transcription induced in the susceptible strain by the presence of an effective antibiotic. To test the ability of the illustrative panel to detect transcriptional changes induced by effective antibiotic the following experiment was conducted. Two strains of Pseudomonas aeruginosa were grown in liquid culture to a density of approximately 1E8 CFU/mL (assessed using a measure of optical density), one strain has a ciprofloxacin MIC of >8 μg/mL (referred to as resistant), the other had a MIC of 0.5 μg/mL (referred to as susceptible). Each culture was then split into equal volume culture tubes and an aliquot removed for testing on the illustrative panel (the zero time point sample). Ciprofloxacin was added to different culture tubes for each strain at either 7.5 μg/mL or 15 μg/mL, and the culture tubes (with and without ciprofloxacin) were returned to the incubator. Samples were removed and tested using individual panels at 10, 30 and 60 minutes for each strain and each condition (+/−ciprofloxacin). The Cp data for each assay acquired from the illustrative panel were normalized on a per pouch basis using either the total RNA signal or the signal from a group of four control genes (bamA, rpoD, prsL, and pal). Normalization was conducted by calculating the geometric mean of the Cp data for the selected targets (either all RNA targets or the four control genes) for each pouch, and then calculating the distance of each assay Cp from the geometric mean (termed the relative expression level). The data from both normalization approaches provided essentially equivalent results, and only the data using total RNA are shown in FIGS. 9A-B.

FIGS. 9A-9B present the relative expression level for the assay target lexA in both the resistant (FIG. 9A) and susceptible strain (FIG. 9B) when exposed to zero, 7.5, or 15 μg/mL ciprofloxacin at 10, 30 and 60 minutes of time. The data are presented as a box plot with outlier points shown in black. The relative expression of lexA in the resistant strain in the absence of ciprofloxacin (FIG. 9A, 0 ciprofloxacin points) did not change when the strain was exposed to, either 7.5 or 15 μg/mL of ciprofloxacin (FIG. 9A compare 7.5 and 15 μg/mL groups to the zero group). For the susceptible strain (FIG. 9B), the data appear quite different. In the susceptible strain, the data show a clear time dependent induction of the lexA transcript in the presence of ciprofloxacin (compare the 0 ciprofloxacin grouping to either the 7.5 or 15 μg/mL ciprofloxacin groupings). The relative expression scale (geometric mean normalized Cp) was derived directly from Cp values; thus lower values indicate higher inputs to the panel. These data demonstrate that the induction of transcription in response to an effective antibiotic may be used to determine antibiotic susceptibility, illustratively using the system described above. The clear time dependent response to ciprofloxacin as well as the magnitude of the induction seen for the lexA target observed using the illustrative panel match nearly exactly the time dependence and magnitude of induction of the lexA gene observed in this susceptible strain, as determined in an independent RNA sequencing experiment.

For generic antibiotic resistance genes, the Cp obtained from a +dsDNAse +RT pouch may be used to identify whether a sample is susceptible or resistant to antibiotics. As discussed above in Example 1, it is expected that incubation of the bacterial sample in the presence of an antibiotic prior to loading into the pouch would result in a shift in Cp for specific antibiotic resistance genes. Illustratively, the bacterial sample may be incubated in a vessel, illustratively, a loading vial as described in U.S. Patent Application No. 2014-0283945, for 10 minutes prior to loading into pouch 510, although other devices and lengths of time for incubation may be desired.

The above demonstrates that mRNA detection and quantification can be done in a pouch similar to pouch 510. The data presented in Example 1 demonstrate the feasibility of using a measure of cellular RNA concentration of generic antibiotic resistance genes and/or specific antibiotic resistance genes as a test for antibiotic resistance. Several embodiments for a bacterial response panel are envisioned. In one embodiment, a single species of bacteria is tested for sensitivity against multiple drugs in a single pouch. In such an embodiment, at least one specific antibiotic resistance gene would be needed for each drug tested. The sample could be incubated against all of the antibiotics in one mixture, or separate aliquots could be incubated against individual antibiotics. In another embodiment, one antibiotic would be tested for susceptibility among a number of bacteria known to have resistance to that antibiotic, where each different species or strain would have one or more targets specific to that species or strain, illustratively, reporting on the presence of the specific species or strain, along with whether the species or strain that is present is also sensitive or resistant to that antibiotic. It is understood that either or both embodiments may be performed using a closed system approach, such as pouch 510, or may be performed using any suitable instrumentation, as is known in the art.

In another embodiment, the minimum inhibitory concentration (MIC) of an antibiotic for a bacterium may be determined by using a pouch 510 that is +DNAse+RT, illustratively where a known amount of the sample is incubated with a known standard concentration of antibiotic for a specific period of time prior to injecting the sample into the pouch and quantifying the amount of mRNA in the sample as a function of Cp. Illustratively, the incubation can be 10 minutes or 30 minutes, although other incubation times may be used. Also illustratively, the breakpoint concentration may be used as the standard concentration, although other concentrations may be chosen. Many of the genes would be specific antibiotic resistance genes, although other genes may be used. For each strain and antibiotic, a different pattern of expression will be seen relative to and is reflective (indicative) of the MIC of the antibiotic. Illustratively, the MIC may be reported as a result of the quantitative output, as discussed above. In another embodiment, a fingerprint of Cps for each of the individual genes may be used to distinguish or compare strains (see, e.g. U.S. Pat. No. 9,200,329, Example 4, herein incorporated by reference).

Similarly, the MIC for multiple antibiotics could be tested in a single pouch 510. Illustratively, aliquots of a sample could be incubated with several antibiotics in separate vessels, each as described above, and then pooled before injection into the pouch. It is understood that, if incubated and combined in a single vessel, a combination of antibiotics may have a synergistic effect on the bacteria present and may give a different pattern of expression than if the sample is aliquoted into several vessels for separate incubation. Either separate or combined incubation may be desired. The output result would be a susceptibility and/or MIC for each antibiotic, which could help the clinician to select an appropriate treatment.

### Example 3

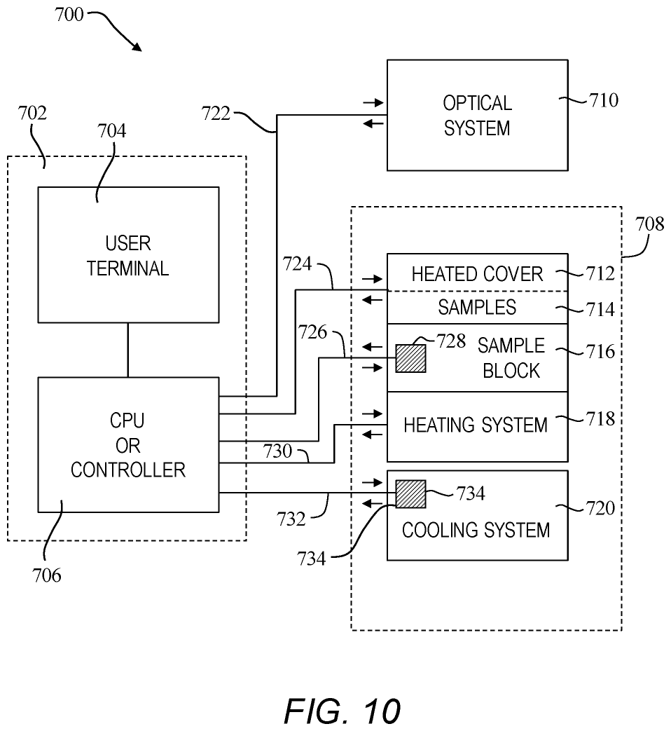

While reference is made herein to the FilmArray system. Other systems are suitable for the methods used herein. Certain embodiments of the present invention may also involve or include a PCR system configured to calls from amplification curves or melting curves or a combination thereof. Illustrative examples are described in U.S. Pat. No. 8,895,295, already incorporated by reference, for use with pouch 510 or similar embodiments. However, it is understood that the embodiments described in U.S. Pat. No. 8,895,295 are illustrative only and other systems may be used according to this disclosure. For example, referring to FIG. 10, a block diagram of an illustrative system 700 that includes control element 702, a thermocycling element 708, and an optical element 710 according to exemplary aspects of the disclosure is shown.

In at least one embodiment, the system may include at least one PCR reaction mixture housed in sample vessel 714. In certain embodiments, the sample vessel 714 may include a PCR reaction mixture configured to permit and/or effect amplification of a template nucleic acid. Certain illustrative embodiments may also include at least one sample block or chamber 716 configured to receive the at least one sample vessel 714. The sample vessel 714 may include any plurality of sample vessels in individual, strip, plate, or other format, and, illustratively, may be provided as or received by a sample block or chamber 716.

One or more embodiments may also include at least one sample temperature controlling device 718 and/or 720 configured to manipulate and/or regulate the temperature of the sample(s). Such a sample temperature controlling device may be configured to raise, lower, and/or maintain the temperature of the sample(s). In one example, sample controlling device 718 is a heating system and sample controlling device 720 is a cooling system. Illustrative sample temperature controlling devices include (but are not limited to) heating and/or cooling blocks, elements, exchangers, coils, radiators, refrigerators, filaments, Peltier devices, forced air blowers, handlers, vents, distributors, compressors, condensers, water baths, ice baths, flames and/or other combustion or combustible forms of heat, hot packs, cold packs, dry ice, dry ice baths, liquid nitrogen, microwave- and/or other wave-emitting devices, means for cooling, means for heating, means for otherwise manipulating the temperature of a sample, and/or any other suitable device configured to raise, lower, and/or maintain the temperature of the sample(s).

The illustrative PCR system 700 also includes an optical system 710 configured to detect an amount of fluorescence emitted by the sample 714 (or a portion or reagent thereof). Such an optical system 710 may include one or more fluorescent channels, as are known in the art, and may simultaneously or individually detect fluorescence from a plurality of samples.

At least one embodiment of the PCR system may further include a CPU 706 programmed or configured to operate, control, execute, or otherwise advance the heating system 718 and cooling system 720 to thermal cycle the PCR reaction mixture, illustratively while optical system 710 collects fluorescent signal. CPU 706 may then generate an amplification curve, a melting curve, or any combination, which may or may not be printed, displayed on a screen of the user terminal 704, or otherwise outputted. Optionally, a positive, negative, or other call may be outputted based on the amplification and/or melting curve for example on the screen of the user terminal 704. Optionally, only the calls are outputted, illustratively, one call for each target tested.

The CPU 706 may include a program memory, a microcontroller or a microprocessor (MP), a random-access memory (RAM), and an input/output (I/O) circuit, all of which are interconnected via an address/data bus. The program memory may include an operating system such as Microsoft Windows®, OS X®, Linux®, Unix®, etc. In some embodiments, the CPU 706 may also include, or otherwise be communicatively connected to, a database or other data storage mechanism (e.g., one or more hard disk drives, optical storage drives, solid state storage devices, etc.). The database may include data such as melting curves, annealing temperatures, denaturation temperatures, and other data necessary to generate and analyze melting curves. The CPU 706 may include multiple microprocessors, multiple RAMS, and multiple program memories as well as a number of different types of I/O circuits. The CPU 706 may implement the RAM(s) and the program memories as semiconductor memories, magnetically readable memories, and/or optically readable memories, for example.

The microprocessors may be adapted and configured to execute any one or more of a plurality of software applications and/or any one or more of a plurality of software routines residing in the program memory, in addition to other software applications. One of the plurality of routines may include a thermocycling routine which may include providing control signals to the heating system 718 and the cooling system 720 to heat and cool the sample 714 respectively, in accordance with the two-step PCR protocol. Another of the plurality of routines may include a fluorescence routine which may include providing control signals to the optical system 710 to emit a fluorescence signal and detect the amount of fluorescence scattered by the sample 714. Yet another of the plurality of routines may include a sample calling routine which may include obtaining fluorescence data (temperature, fluorescence pairs) from the optical system 710 during the in-cycle temperature adjusting segment for each of N cycles, generating a composite melting curve by combining the fluorescent data from each of the N cycles during the respective in-cycle temperature adjusting segments, analyzing the composite melting curve to make a positive or negative call, and displaying the composite melting curve, individual melting curve, and/or an indication of the call on the user terminal 704.

In some embodiments, the CPU 706 may communicate with the user terminal 704, the heating system 718, the cooling system 720, the optical system 710, and the sample block 716 over a communication network 722-732 via wired or wireless signals and, in some instances, may communicate over the communication network via an intervening wireless or wired device, which may be a wireless router, a wireless repeater, a base transceiver station of a mobile telephony provider, etc. The communication network may be a wireless communication network such as a fourth- or third-generation cellular network (4G or 3G, respectively), a Wi-Fi network (802.11 standards), a WiMAX network, a wide area network (WAN), a local area network (LAN), the Internet, etc. Furthermore, the communication network may be a proprietary network, a secure public Internet, a virtual private network and/or some other type of network, such as dedicated access lines, plain ordinary telephone lines, satellite links, combinations of these, etc. Where the communication network comprises the Internet, data communication may take place over the communication network via an Internet communication protocol. Still further, the communication network may be a wired network where data communication may take place via Ethernet or a Universal Serial Bus (USB) connection.

In some embodiments, the CPU 706 may be included within the user terminal 704. In other embodiments, the CPU 706 may communicate with the user terminal 704 via a wired or wireless connection (e.g., as a remote server) to display individual melting curves, composite melting curves, calls, etc. on the user terminal 704. The user terminal 704 may include a user interface, a communication unit, and a user-input device such as a “soft” keyboard that is displayed on the user interface of the user terminal 704, an external hardware keyboard communicating via a wired or a wireless connection (e.g., a Bluetooth keyboard), an external mouse, or any other suitable user-input device in addition to the CPU 706 or another CPU similar to the CPU 706.

Additional examples of illustrative features, components, elements, and or members of illustrative PCR systems and/or thermal cyclers (thermocyclers) are known in the art and/or described above or in U.S. Patent Application No. 2014-0273181, the entirety of which is herein incorporated by reference.

### REFERENCES

Barczak, A. K., et al. “RNA signatures allow rapid identification of pathogens and antibiotic susceptibilities.” Proceedings of the National Academy of Sciences, vol. 109, no. 16, February 2012, pp. 6217-6222.
Bartholomaus A, Fedyunin I, Feist P, Sin C, Zhang G, Valleriani A, Ignatova Z. 2016 Bacteria differentially regulate mRNA abundance to specifically respond to various stresses. Phil. Tans. R. Soc. A374: 20150069.

Described herein are:

1. A method for determining antibiotic resistance of a bacterium in a sample comprising:

(a) incubating the sample with an antibiotic,

(b) isolating RNA from the sample,

(c) reverse-transcribing the RNA for a plurality of genes that each show a different pattern of expression between susceptible and resistant strains,

(d) amplifying targets from the plurality of genes that each show a different pattern of expression between susceptible and resistant strains to generate a plurality of amplified targets,

(e) quantifying each of the plurality of amplified targets to provide a plurality of quantified amplified targets and to generate a value indicative of antibiotic susceptibility, and

(f) determining antibiotic resistance from the value indicative of antibiotic susceptibility.

2. The method of claim 1, wherein

step (c) further includes reverse-transcribing the RNA for a reference gene,

step (d) further includes amplifying the reference gene,

step (e) further includes quantifying the reference gene to generate a reference value, and

step (f) includes comparing the reference value to the plurality of quantified amplified targets.

3. The method of clause 2, wherein

step (c) further includes reverse-transcribing the RNA for at least one additional reference gene,

step (d) further includes amplifying at least one additional target from the at least additional reference gene, and

step (e) includes quantifying the at least one additional reference gene to use in generating the reference value.

4. The method of any one of clauses 2-3, further comprising

calculating a value from the reference value for each of the plurality of quantified amplified genes wherein the value is selected from a real value or an absolute value, wherein the value indicative of antibiotic susceptibility is an output (e.g., a mathematical output) obtained using the value for each of the plurality of quantified amplified genes, optionally wherein the output (e.g., mathematical output) is a sum of the absolute value for each of the plurality of quantified amplified genes.

5. The method of any one of clauses 1-4, wherein the plurality of genes includes a generic antibiotic resistance gene.

6. The method of any one of clauses 1-5, wherein the plurality of genes includes a specific antibiotic resistance gene.

7. The method of any one of clauses 1-6, wherein the plurality of genes includes a generic antibiotic resistance gene and a specific antibiotic resistance gene.

8. The method of any one of clauses 1-7, wherein the bacterium is one of a plurality of bacteria known to have resistance to the antibiotic.

9. The method of any one of clauses 1-8, wherein step (a) includes incubating the sample with a plurality of additional antibiotics, wherein a first set of the plurality of genes is relevant to the antibiotic, and additional sets of the plurality of genes are relevant to the additional antibiotics.

10. The method of any one of clauses 1-9, further comprising removing DNA from the sample prior to step (c).

11. The method of any one of clauses 1-10, wherein the plurality of amplified targets from the plurality of genes includes one or more amplicons of at least 300 bp.

12. The method of any one of clauses 1-11, wherein each the plurality of amplified targets results in an amplicon of at least 300 bp.

13. The method of any one of clauses 1-12, wherein the plurality of amplified targets from the plurality of genes includes one or more amplicons of at least 500 bp.

14. The method of any one of clauses 10-13, wherein removing the DNA includes a digestion by a DNAse lasting no more than 10 minutes.

15. A method for determining antibiotic resistance of a bacterium in a sample comprising:

(a) incubating the sample with an antibiotic,

(b) isolating RNA from the sample,

(c) reverse-transcribing the RNA for a gene that shows a different pattern of expression between susceptible and resistant strains,

(d) amplifying a target from the gene that shows the different pattern of expression between susceptible and resistant strains to generate an amplified target,

(e) quantifying the amplified target to generate a value indicative of antibiotic susceptibility, and

(f) determining antibiotic resistance from the value indicative of antibiotic susceptibility.

16. A container for determining antibiotic resistance of a bacterium in a sample comprising:

a first-stage reaction zone comprising a first-stage reaction blister comprising a plurality of pairs of primers for reverse-transcription and amplification of a plurality of genes that each show a different pattern of expression between susceptible and resistant strains, and

a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show a different pattern of expression between susceptible and resistant strains, the second-stage reaction zone configured for thermal cycling all of the plurality of second-stage reaction chambers.

17. A device for analyzing a sample, comprising:

an opening configured to receive a container, the container comprising a first-stage reaction zone comprising a plurality of pairs of primers for reverse-transcription and amplification of a plurality of genes that each show a different pattern of expression between susceptible and resistant strains or a reference gene, and

a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that show the different pattern of expression between susceptible and resistant strains or the reference gene, the plurality of second-stage reaction chambers further comprising a detectable label that produces a signal indicative of an amount of amplification,

a first heater for controlling temperature of the first-stage reaction zone,

a second heater for thermal cycling the second-stage reaction zone,

a detection device configured to detect the signal in each of the second-stage reaction chambers, and

a CPU configured to determine a Cp for each of the plurality of genes that each show the different pattern of expression between susceptible and resistant strains and the reference gene, and configured to output a value for each of the plurality of genes that show the different pattern of expression between susceptible and resistant strains and the reference gene, wherein the value is a ΔCp or absolute value of a ΔCp for each of the plurality of genes that each show the different pattern of expression between susceptible and resistant strains, and wherein the CPU is configured to determine antibiotic resistance from the values for each of the plurality of genes that show the different pattern of expression between susceptible and resistant strains.

18. A method for determining the minimal inhibitory concentration (MIC) of an antibiotic towards a bacterium in a sample comprising:

(a) incubating an aliquot of the sample with a known standard concentration of the antibiotic,

(b) isolating RNA from the aliquot of the sample, the RNA comprising a gene that shows a quantitatively different level of expression relative to the MIC of the antibiotic,

(c) reverse transcribing the RNA for the gene,

(d) amplifying a target of the gene to generate an amplified target,

(e) quantifying the amplified target to provide a quantified amplified target and to generate a value indicative of the MIC, and

(f) reporting the MIC as a result of the quantitative output for the gene.

19. The method of clause 18, wherein the known standard concentration of the antibiotic is a breakpoint concentration.

20. The method of clause 18 or 19, wherein the RNA from the sample comprises a plurality of additional genes that show a quantitatively different level of expression relative to the MIC of the antibiotic, the method further comprising:

reverse transcribing RNA for the plurality of additional genes,

amplifying targets from the plurality of additional genes to generate a plurality of amplified targets from the plurality of additional genes,

quantifying each of the plurality of amplified targets from the plurality of additional genes to generate a value indicative of the MIC for each of the plurality of additional genes, and

reporting the MIC as a combination of the quantitative output for the gene and the plurality of additional genes.

21. The method of any one of clauses 18-20, wherein

step (a) includes incubating a plurality of additional aliquots of the sample each with a known standard concentration of an additional antibiotic,

pulling the aliquot of the sample and the plurality of additional aliquots of the sample prior to step (b),

reverse transcribing RNA for a plurality of genes for each additional antibiotic,

amplifying targets from the plurality of genes for each of the additional antibiotics to generate a plurality of amplified targets from the plurality of genes for each of the additional antibiotics,

quantifying each of the plurality of amplified targets from the plurality of genes for each additional antibiotic to generate a value indicative of the MIC for each of the plurality of genes for each additional antibiotic, and

reporting the MIC for each additional antibiotic as a combination of the quantitative output for the plurality of genes for each additional antibiotic.

22. The method of any one of clauses 18-21, wherein

step (d) further includes amplifying a target from a reference gene,

step (e) further includes quantifying the reference gene to generate a reference value, and

step (f) includes comparing the reference value to the quantified amplified target.

23. The method of any one of clauses 18-22, wherein the gene is a specific antibiotic resistance gene.

24. The method of any one of clauses 18-23, further comprising removing DNA from the sample prior to step (c).

25. The method of any one of clauses 18-24, wherein the amplified target includes one or more amplicons of at least 300 bp.

26. The method of any one of clauses 18-25, wherein each the amplified target results in an amplicon of at least 300 bp.

27. The method of any one of clauses 18-26, wherein the amplified target includes one or more amplicons of at least 500 bp.

28. The method of any one of clauses 24-27, wherein removing the DNA includes a digestion by a dsDNAse lasting no more than 10 minutes.

29. The method of any one of clauses 1-15 or 18-28, wherein one or more steps of the method is carried out using and/or carried out in the container of clause 16, optionally wherein an isolating step, reverse-transcribing step, and/or amplifying step is carried out using and/or carried out in the container of clause 16.

30. The method of any one of clauses 1-15 or 18-28, wherein one or more steps of the method is carried out using and/or carried out in the device of clause 17, optionally wherein an isolating step, reverse-transcribing step, and/or amplifying step is carried out using and/or carried out in the device of clause 17.

31. A method for determining an effect of an antibiotic on a bacterium in a sample comprising:

(a) incubating the sample with the antibiotic,

(b) isolating RNA from the sample,

(c) reverse-transcribing the RNA for a plurality of genes,

(d) amplifying targets from the plurality of genes to generate a plurality of amplified targets, and

(e) comparing the amplified targets with amplified targets from another sample of the bacterium that has not been incubated with the antibiotic.

32. The method of clause 32, further incorporating the steps of any of clauses 2-14.

33. Use of the container of clause 16 in a method of any one of clauses 1-15, 18-28, or 31-32.

34. Use of the device of clause 17 in a method of any one of claim 1-15, 18-28, or 31-32.

Although the invention has been described in detail with reference to preferred embodiments, variations and modifications exist within the scope and spirit of the invention as described and defined in the following claims.

1. A method for determining antibiotic susceptibility of a bacterium in a sample comprising:
(a) incubating the sample with an antibiotic,
(b) isolating RNA from the sample,
(c) reverse-transcribing the RNA for a plurality of genes that each show a different pattern of expression between susceptible and resistant strains,
(d) amplifying targets from the plurality of genes that each show a different pattern of expression between susceptible and resistant strains to generate a plurality of amplified targets,
(e) quantifying each of the plurality of amplified targets from the plurality of genes to provide a plurality of quantified amplified targets and to generate a value indicative of antibiotic susceptibility, and
(f) determining antibiotic susceptibility from the value indicative of antibiotic susceptibility.

2. The method of claim 1, wherein
step (c) further includes reverse-transcribing the RNA for a reference gene,
step (d) further includes amplifying a target from the reference gene,
step (e) further includes quantifying the reference gene to generate a reference value, and
step (f) includes comparing the reference value to the plurality of quantified amplified targets from the plurality of genes.

3. The method of claim 2, wherein
step (c) further includes reverse-transcribing the RNA for at least one additional reference gene,
step (d) further includes amplifying at least one additional target from the at least one additional reference gene, and
step (e) includes quantifying the at least one additional reference gene to use in generating the reference value.

4. The method of claim 2, further comprising
calculating a value from the reference value for each of the plurality of quantified amplified genes wherein the value is selected from a real value or an absolute value, wherein the value indicative of antibiotic susceptibility is an output obtained using the value for each of the plurality of quantified amplified genes.

5. The method of claim 1, wherein the plurality of genes includes a generic antibiotic resistance gene.

6. The method of claim 1, wherein the plurality of genes includes a specific antibiotic resistance gene.

7. The method of claim 1, wherein the plurality of genes includes a generic antibiotic resistance gene and a specific antibiotic resistance gene.

8. The method of claim 1, wherein the bacterium is one of a plurality of bacteria known to have susceptibility to the antibiotic.

9. The method of claim 1, wherein step (a) includes incubating the sample with a mixture of the antibiotic and additional antibiotics, wherein a first set of the plurality of genes is relevant to the antibiotic, and additional sets of the plurality of genes are relevant to each of the additional antibiotics.

10. The method of claim 1, further comprising removing DNA from the sample prior to step (c) by using a digestion by a DNAse lasting no more than 10 minutes.

11. The method of claim 10, wherein the plurality of amplified targets from the plurality of genes includes one or more amplicons of at least 300 bp.

12. The method of claim 10, wherein each the plurality of amplified targets results in an amplicon of at least 300 bp.

13. The method of claim 10, wherein the plurality of amplified targets from the plurality of genes includes one or more amplicons of at least 500 bp.

14.-15. (canceled)

16. A container for determining antibiotic susceptibility of a bacterium in a sample comprising
a first-stage reaction zone comprising a first-stage reaction blister comprising a plurality of pairs of primers for reverse-transcription and amplification of a plurality of genes that each show a different pattern of expression between susceptible and resistant strains, and
a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show a different pattern of expression between susceptible and resistant strains, the second-stage reaction zone configured for thermal cycling all of the plurality of second-stage reaction chambers.

17. A device for analyzing a sample, comprising:
an opening configured to receive a container, the container comprising a first-stage reaction zone comprising a plurality of pairs of primers for reverse-transcription and amplification of a plurality of genes that each show a different pattern of expression between susceptible and resistant strains or a reference gene, and
a second-stage reaction zone fluidly connected to the first-stage reaction zone, the second-stage reaction zone comprising a plurality of second-stage reaction chambers, each second-stage reaction chamber comprising a pair of primers for further amplification of the plurality of genes that each show the different pattern of expression between susceptible and resistant strains or the reference gene, the plurality of second-stage reaction chambers further comprising a detectable label that produces a signal indicative of an amount of amplification,
a first heater for controlling temperature of the first-stage reaction zone,
a second heater for thermal cycling the second-stage reaction zone,
a detection device configured to detect the signal in each of the second-stage reaction chambers, and
a CPU configured to determine a Cp for each of the plurality of genes that each show the different pattern of expression between susceptible and resistant strains and the reference gene, and configured to output a value for each of the plurality of genes that each show the different pattern of expression between susceptible and resistant strains, wherein the value is a ΔCp or absolute value of a ΔCp for each of the plurality of genes that each show the different pattern of expression between susceptible and resistant strains, and wherein the CPU is configured to determine antibiotic susceptibility from the values for each of the plurality of genes that each show the different pattern of expression between susceptible and resistant strains.

18. A method for determining the minimal inhibitory concentration (MIC) of an antibiotic towards a bacterium in a sample comprising:
(a) incubating an aliquot of the sample with a known standard concentration of the antibiotic,
(b) isolating RNA from the aliquot of the sample, the RNA comprising a gene that shows a quantitatively different level of expression relative to the MIC of the antibiotic,
(c) reverse transcribing the RNA for the gene,
(d) amplifying a target of the gene to generate an amplified target,
(e) quantifying the amplified target to provide a quantified amplified target and to generate a value indicative of the MIC, and
(f) reporting the MIC as a result of the quantitative output for the gene.

19. The method of claim 18, wherein the known standard concentration of the antibiotic is a breakpoint concentration.

20. The method of claim 18, wherein the RNA from the sample comprises a plurality of additional genes that show a quantitatively different level of expression relative to the MIC of the antibiotic, the method further comprising:
reverse transcribing RNA for the plurality of additional genes,
amplifying targets from the plurality of additional genes to generate a plurality of amplified targets from the plurality of additional genes,
quantifying each of the plurality of amplified targets from the plurality of additional genes to generate a value indicative of the MIC for each of the plurality of additional genes, and
reporting the MIC as a combination of the quantitative output for the gene and the plurality of additional genes, wherein
step (a) includes incubating a plurality of additional aliquots of the sample each with a known standard concentration of an additional antibiotic,
pulling the aliquot of the sample and the plurality of additional aliquots of the sample prior to step (b),
reverse transcribing RNA for a plurality of genes for each additional antibiotic,
amplifying targets from the plurality of genes for each of the additional antibiotics to generate a plurality of amplified targets from the plurality of genes for each of the additional antibiotics,
quantifying each of the plurality of amplified targets from the plurality of genes for each additional antibiotic to generate a value indicative of the MIC for each of the plurality of genes for each additional antibiotic, and
reporting the MIC for each additional antibiotic as a combination of the quantitative output for the plurality of genes for each additional antibiotic.

21. (canceled)

22. The method of claim 18, wherein
step (d) further includes amplifying a target from a reference gene,
step (e) further includes quantifying the reference gene to generate a reference value, and
step (f) includes comparing the reference value to the quantified amplified target.

23. The method of claim 18, wherein the gene is a specific antibiotic resistance gene.

24. The method of claim 18, further comprising removing DNA from the sample prior to step (c) by including a digestion by a dsDNAse lasting no more than 10 minutes.

25. The method of claim 24, wherein the amplified target includes one or more amplicons of at least 300 bp.

26. The method of claim 24, wherein each the amplified target results in an amplicon of at least 300 bp.

27. The method of claim 24, wherein the amplified target includes one or more amplicons of at least 500 bp.

28.-29. (canceled)

30. The method of claim 1 wherein steps (b) through (d) are performed in a sealed container.

31. The method of claim 10 wherein the step of removing DNA from the sample and steps (b) through (d) are all performed in a sealed container.

32. The method of claim 1 wherein step (a) takes place in 10, 30, or 60 minutes.

33. The method of claim 9 wherein step (a) takes place in 10, 30, or 60 minutes.
